SEQUENCE LISTING (22 sequences)

SEQ ID NO: 1 - protein, 176 aa, Escherichia coli
MSLLNVPAGKDLPEDIYVVIEIPANADPIKYEIDKESGALFVDRFMSTAMFYPCNYGYINHTLSLDGDPVDVLVPTPYPLQPGSVIRCRPVGVLKMTDEAGEDAKLVAVPHSKLSKEYDHIKDVNDLPELLKAQIAHFFEHYKDLEKGKWVKVEGWENAEAAKAEIVASFERAKNK

SEQ ID NO: 2 - protein, 192 aa, Artificial Sequence
  Mutated Escherichia coli PPase_ C54S,C88A,177(GGGGS)3C
MSLLNVPAGKDLPEDIYVVIEIPANADPIKYEIDKESGALFVDRFMSTAMFYPSNYGYINHTLSLDGDPVDVLVPTPYPLQPGSVIRARPVGVLKMTDEAGEDAKLVAVPHSKLSKEYDHIKDVNDLPELLKAQIAHFFEHYKDLEKGKWVKVEGWENAEAAKAEIVASFERAKNKGGGGSGGGGSGGGGSC

SEQ ID NO: 3 - protein, 192 aa, Artificial Sequence
  Mutated Escherichia coli PPase_ C54A,C88A,177(GGGGS)3C
MSLLNVPAGKDLPEDIYVVIEIPANADPIKYEIDKESGALFVDRFMSTAMFYPANYGYINHTLSLDGDPVDVLVPTPYPLQPGSVIRARPVGVLKMTDEAGEDAKLVAVPHSKLSKEYDHIKDVNDLPELLKAQIAHFFEHYKDLEKGKWVKVEGWENAEAAKAEIVASFERAKNKGGGGSGGGGSGGGGSC

SEQ ID NO: 4 - protein, 192 aa, Artificial Sequence
  Mutated Escherichia coli PPase_ C54A,C88S,177(GGGGS)3C
MSLLNVPAGKDLPEDIYVVIEIPANADPIKYEIDKESGALFVDRFMSTAMFYPANYGYINHTLSLDGDPVDVLVPTPYPLQPGSVIRSRPVGVLKMTDEAGEDAKLVAVPHSKLSKEYDHIKDVNDLPELLKAQIAHFFEHYKDLEKGKWVKVEGWENAEAAKAEIVASFERAKNKGGGGSGGGGSGGGGSC

SEQ ID NO: 5 - protein, 192 aa, Artificial Sequence
  Mutated Escherichia coli PPase_ C54S,C88S,177(GGGGS)3C
MSLLNVPAGKDLPEDIYVVIEIPANADPIKYEIDKESGALFVDRFMSTAMFYPSNYGYINHTLSLDGDPVDVLVPTPYPLQPGSVIRSRPVGVLKMTDEAGEDAKLVAVPHSKLSKEYDHIKDVNDLPELLKAQIAHFFEHYKDLEKGKWVKVEGWENAEAAKAEIVASFERAKNKGGGGSGGGGSGGGGSC

SEQ ID NO: 6 - protein, 177 aa, Artificial Sequence
  Mutated Escherichia coli PPase_ C54S,C88A,177(GGGGS)3C
MSLLNVPAGKDLPEDIYVVIEIPANADPIKYEIDKESGALFVDRFMSTAMFYPSNYGYINHTLSLDGDPVDVLVPTPYPLQPGSVIRARPVGVLKMTDEAGEDAKLVAVPHSKLSKEYDHIKDVNDLPELLKAQIAHFFEHYKDLEKGKWVKVEGWENAEAAKAEIVASFERAKNKC

SEQ ID NO: 7 - protein, 177 aa, Artificial Sequence
  Mutated Escherichia coli PPase_ C54A,C88A,177C
MSLLNVPAGKDLPEDIYVVIEIPANADPIKYEIDKESGALFVDRFMSTAMFYPANYGYINHTLSLDGDPVDVLVPTPYPLQPGSVIRARPVGVLKMTDEAGEDAKLVAVPHSKLSKEYDHIKDVNDLPELLKAQIAHFFEHYKDLEKGKWVKVEGWENAEAAKAEIVASFERAKNKC

SEQ ID NO: 8 - protein, 177 aa, Artificial Sequence
  Mutated Escherichia coli PPase_ C54A,C88S,177C
MSLLNVPAGKDLPEDIYVVIEIPANADPIKYEIDKESGALFVDRFMSTAMFYPANYGYINHTLSLDGDPVDVLVPTPYPLQPGSVIRSRPVGVLKMTDEAGEDAKLVAVPHSKLSKEYDHIKDVNDLPELLKAQIAHFFEHYKDLEKGKWVKVEGWENAEAAKAEIVASFERAKNKC

SEQ ID NO: 9 - protein, 182 aa, Artificial Sequence
  Mutated Escherichia coli PPase_ C54A,C88A,177GGGGGC
MSLLNVPAGKDLPEDIYVVIEIPANADPIKYEIDKESGALFVDRFMSTAMFYPANYGYINHTLSLDGDPVDVLVPTPYPLQPGSVIRARPVGVLKMTDEAGEDAKLVAVPHSKLSKEYDHIKDVNDLPELLKAQIAHFFEHYKDLEKGKWVKVEGWENAEAAKAEIVASFERAKNKGGGGGC

SEQ ID NO: 10 - protein, 175 aa, Thermus aquaticus
MANLKSLPVGKNAPQVVHMVIEVPRGSGNKYEYDPELGVVKLDRVLPGAQFYPGDYGFIPSTLAEDGDPLDGLILSTYPLLPGVVVEVRPVGLLLMEDEKGGDAKILGVVAEDQRLDHIQDIGDVPEGVKQEIQHFFETYKALEAKKGKWVRVTGWRDRQAALEEIQAAIARYGR

SEQ ID NO: 11 - protein, 185 aa, Artificial Sequence
  Mutated inorganic pyrophosphatase [Thermus aquaticus]176GGGGSGGGGC
MANLKSLPVGKNAPQVVHMVIEVPRGSGNKYEYDPELGVVKLDRVLPGAQFYPGDYGFIPSTLAEDGDPLDGLILSTYPLLPGVVVEVRPVGLLLMEDEKGGDAKILGVVAEDQRLDHIQDIGDVPEGVKQEIQHFFETYKALEAKKGKWVRVTGWRDRQAALEEIQAAIARYGRGGGGSGGGGC

SEQ ID NO: 12 - protein, 181 aa, Artificial Sequence
  Mutated inorganic pyrophosphatase [Thermus aquaticus]176GGGGGC
MANLKSLPVGKNAPQVVHMVIEVPRGSGNKYEYDPELGVVKLDRVLPGAQFYPGDYGFIPSTLAEDGDPLDGLILSTYPLLPGVVVEVRPVGLLLMEDEKGGDAKILGVVAEDQRLDHIQDIGDVPEGVKQEIQHFFETYKALEAKKGKWVRVTGWRDRQAALEEIQAAIARYGRGGGGGC

SEQ ID NO: 13 - protein, 176 aa, Artificial Sequence
  Mutated inorganic pyrophosphatase [Thermus aquaticus]176C
MANLKSLPVGKNAPQVVHMVIEVPRGSGNKYEYDPELGVVKLDRVLPGAQFYPGDYGFIPSTLAEDGDPLDGLILSTYPLLPGVVVEVRPVGLLLMEDEKGGDAKILGVVAEDQRLDHIQDIGDVPEGVKQEIQHFFETYKALEAKKGKWVRVTGWRDRQAALEEIQAAIARYGRC

SEQ ID NO: 14 - protein, 191 aa, Artificial Sequence
  Mutated inorganic pyrophosphatase [Thermus aquaticus]176(GGGGS)3C
MANLKSLPVGKNAPQVVHMVIEVPRGSGNKYEYDPELGVVKLDRVLPGAQFYPGDYGFIPSTLAEDGDPLDGLILSTYPLLPGVVVEVRPVGLLLMEDEKGGDAKILGVVAEDQRLDHIQDIGDVPEGVKQEIQHFFETYKALEAKKGKWVRVTGWRDRQAALEEIQAAIARYGRGGGGSGGGGSGGGGSC

SEQ ID NO: 15 - protein, 175 aa, Thermus thermophilus
MANLKSLPVGDKAPEVVHMVIEVPRGSGNKYEYDPDLGAIKLDRVLPGAQFYPGDYGFIPSTLAEDGDPLDGLVLSTYPLLPGVVVEVRVVGLLLMEDEKGGDAKVIGVVAEDQRLDHIQDIGDVPEGVKQEIQHFFETYKALEAKKGKWVKVTGWRDRKAALEEVRACIARYKG

SEQ ID NO: 16 - protein, 176 aa, Artificial Sequence
  Mutated inorganic pyrophosphatase [Thermus thermophilus]_C169A,176C
MANLKSLPVGDKAPEVVHMVIEVPRGSGNKYEYDPDLGAIKLDRVLPGAQFYPGDYGFIPSTLAEDGDPLDGLVLSTYPLLPGVVVEVRVVGLLLMEDEKGGDAKVIGVVAEDQRLDHIQDIGDVPEGVKQEIQHFFETYKALEAKKGKWVKVTGWRDRKAALEEVRAAIARYKGC

SEQ ID NO: 17 - protein, 176 aa, Artificial Sequence
  Mutated inorganic pyrophosphatase [Thermus thermophilus]_C169S,176C
MANLKSLPVGDKAPEVVHMVIEVPRGSGNKYEYDPDLGAIKLDRVLPGAQFYPGDYGFIPSTLAEDGDPLDGLVLSTYPLLPGVVVEVRVVGLLLMEDEKGGDAKVIGVVAEDQRLDHIQDIGDVPEGVKQEIQHFFETYKALEAKKGKWVKVTGWRDRKAALEEVRASIARYKGC

SEQ ID NO: 18 - protein, 191 aa, Artificial Sequence
  Mutated inorganic pyrophosphatase [Thermus thermophilus]_C169A,176(GGGGS)3C
MANLKSLPVGDKAPEVVHMVIEVPRGSGNKYEYDPDLGAIKLDRVLPGAQFYPGDYGFIPSTLAEDGDPLDGLVLSTYPLLPGVVVEVRVVGLLLMEDEKGGDAKVIGVVAEDQRLDHIQDIGDVPEGVKQEIQHFFETYKALEAKKGKWVKVTGWRDRKAALEEVRAAIARYKGGGGGSGGGGSGGGGSC

SEQ ID NO: 19 - protein, 191 aa, Artificial Sequence
  Mutated inorganic pyrophosphatase [Thermus thermophilus]_C169S, 176(GGGGS)3C
MANLKSLPVGDKAPEVVHMVIEVPRGSGNKYEYDPDLGAIKLDRVLPGAQFYPGDYGFIPSTLAEDGDPLDGLVLSTYPLLPGVVVEVRVVGLLLMEDEKGGDAKVIGVVAEDQRLDHIQDIGDVPEGVKQEIQHFFETYKALEAKKGKWVKVTGWRDRKAALEEVRASIARYKGGGGGSGGGGSGGGGSC

SEQ ID NO: 20 - protein, 181 aa, Artificial Sequnece
  Mutated inorganic pyrophosphatase [Thermus thermophilus]_C169A,176GGGGGC
MANLKSLPVGDKAPEVVHMVIEVPRGSGNKYEYDPDLGAIKLDRVLPGAQFYPGDYGFIPSTLAEDGDPLDGLVLSTYPLLPGVVVEVRVVGLLLMEDEKGGDAKVIGVVAEDQRLDHIQDIGDVPEGVKQEIQHFFETYKALEAKKGKWVKVTGWRDRKAALEEVRAAIARYKGGGGGGC

SEQ ID NO: 21 - protein, 181 aa, Artificial Sequence
  Inorganic pyrophosphatase [Thermus thermophilus]_C169A, 176GGGGGC
MANLKSLPVGDKAPEVVHMVIEVPRGSGNKYEYDPDLGAIKLDRVLPGAQFYPGDYGFIPSTLAEDGDPLDGLVLSTYPLLPGVVVEVRVVGLLLMEDEKGGDAKVIGVVAEDQRLDHIQDIGDVPEGVKQEIQHFFETYKALEAKKGKWVKVTGWRDRKAALEEVRASIARYKGGGGGGC

SEQ ID NO: 22 - RNA, 1653 nt, Artificial sequence
  PPLuc modified coding sequence
auggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccguguga

(19) United States
(12) Patent Application Publication
(10) Pub. No.: US 2019/0177714 A1
(43) Pub. Date: Jun. 13, 2019

(54) IMMOBILIZED INORGANIC PYROPHOSPHATASE (PPASE)

(71) Applicant: CureVac AG, Tübingen (DE)

(72) Inventors: Martin KUNZE, Rottenburg (DE); Felix Niklas HALDER, Karlsruhe (DE); Benyamin YAZDAN PANAH, Tübingen (DE); Tilmann ROOS, Kusterdingen (DE)

(21) Appl. No.: 16/087,297

(22) PCT Filed: Mar. 24, 2016
(86) PCT No.: PCT/EP2016/056615

§ 371 Date: Sep. 21, 2018

(51) Int. Cl. C12N 11/06; C12N 11/08; C12P 19/34; C12M 1/40; C12N 9/14

(52) U.S. Cl. CPC C12N 11/06; C12N 11/08; C12Y 306/01001; C12M 21/18; C12N 9/14; C12P 19/34

(57) ABSTRACT

The present invention relates to an inorganic pyrophosphatase (PPase), methods of producing the same and uses thereof. Further disclosed are an enzyme reactor and a kit comprising the PPase.

### FIELD OF THE INVENTION

The present invention relates to an inorganic pyrophosphatase (PPase), methods of producing the same and uses thereof. Further disclosed are an enzyme reactor and a kit comprising the PPase.

### BACKGROUND OF THE INVENTION

Therapeutic ribonucleic acid (RNA) molecules represent an emerging class of drugs. RNA-based therapeutics include messenger-RNA (mRNA) molecules encoding antigens for use as vaccines (Fotin-Mleczek et al. (2012) J. Gene Med. 14(6):428-439). In addition, it is envisioned to use RNA molecules for replacement therapies, e.g. providing missing proteins such as growth factors or enzymes to patients. Furthermore, the therapeutic use of non-coding immunostimulatory RNA molecules and other non-coding RNAs such as microRNAs, small interfering RNAs (siRNAs), Clustered Regularly Interspaced Short Palindromic Repeats (CRISPR/Cas9) guide RNAs, and long non-coding RNAs is considered.

For the successful development of RNA therapeutics, the production of RNA molecules as active pharmaceutical ingredients must be efficient in terms of yield, quality, safety and costs, especially when RNA is produced at a large scale.

In the art, straightforward processes for the recombinant production of RNA molecules in preparative amounts have been developed in a process called “RNA in vitro transcription”. The term “RNA in vitro transcription” relates to a process wherein RNA is synthesized in a cell-free system (in vitro). RNA is commonly obtained by enzymatic DNA dependent in vitro transcription of an appropriate DNA template, which is often a linearized plasmid DNA template. The promoter for controlling RNA in vitro transcription can be any promoter for any DNA dependent RNA polymerase. Particular examples of DNA dependent RNA polymerases are the bacteriophage enzymes T7, T3, and SP6 RNA polymerases.

Methods for RNA in vitro transcription are known in the art (see for example Geall et al. (2013) Semin. Immunol. 25(2): 152-159; Brunelle et al. (2013) Methods Enzymol. 530: 101-14). Reagents used in said methods may include a linear DNA template with a promoter sequence that has a high binding affinity for its respective RNA polymerase; ribonucleoside triphosphates (NTPs) for the four bases (adenine, cytosine, guanine and uracil); a cap analog (e.g., m7G(5′)ppp(5′)G (m7G)); other modified nucleotides; DNA-dependent RNA polymerase (e.g., T7, T3 or SP6 RNA polymerase); ribonuclease (RNase) inhibitor to inactivate contaminating RNase; MgCl₂, which supplies Mg²⁺ as a cofactor for the RNA polymerase; antioxidants (e.g. DTT); polyamines such as spermidine; and a buffer to maintain a suitable pH value.

In addition to the above mentioned compounds, the enzyme inorganic pyrophosphatase (PPase) has been widely used in methods wherein nucleic acid molecules are produced, such as RNA in vitro transcription reactions but also in DNA sequencing reactions and cDNA transcription reactions because the addition of PPase increases transcription yields and minimizes the effect of variation of magnesium concentration (see for example Cunningham P. R. and Ofengand J. (1990) Biotechniques 9(6): 713-714.). The addition of PPase to the reaction mixture (e.g., to RNA in vitro transcription reactions using DNA dependent RNA polymerases or to cDNA in vitro transcription using RNA dependent DNA polymerases) catalyzes the hydrolysis of inorganic pyrophosphate and thus prevents its direct inhibitory action of the transcription enzyme. Later in the reaction, when the nucleotide levels have been depleted, removing pyrophosphate serves to free Mg⁺⁺ and promotes Mg⁺⁺-NTP formation and thus allows polymer synthesis to occur with sub-saturating levels of Mg⁺⁺.

RNA in vitro transcription reactions are typically performed as batch reactions in which all components are combined and then incubated to allow the synthesis of RNA molecules until the reaction terminates. In addition, fed-batch reactions were developed to increase the efficiency of the RNA in vitro transcription reaction (Kern et al. (1997) Biotechnol. Prog. 13: 747-756; Kern et al. (1999) Biotechnol. Prog. 15: 174-184). In a fed-batch system, all components are combined, but then additional amounts of some of the reagents are added over time (e.g., NTPs, MgCl₂) to maintain constant reaction conditions.

Moreover, the use of a bioreactor (transcription reactor) for the synthesis of RNA molecules by in vitro transcription has been reported (WO 95/08626). The bioreactor is configured such that reactants are delivered via a feed line to the reactor core and RNA products are removed by passing through an ultrafiltration membrane (having a nominal molecular weight cut-off, e.g., 100,000 daltons (Da)) to the exit stream.

To date, the removal of pyrophosphate (PPᵢ, P₂O₇⁴) in transcription reactions is performed using PPases which are in solution together with all other reaction components. After the transcription reaction, the transcripts are separated and PPases are discarded. The state of the art transcription reaction thus requires large amounts of PPases and a more advanced purification procedure to remove said PPases from the end product (e.g., RNA or cDNA).

In large scale nucleic acid production, it would be a major economic advantage to use insolubilized or immobilized PPase enzymes. In particular, if the transcription reactions are intended to be performed in continuous flow or in bioreactors as explained above, an immobilization of the involved enzymes, particularly of PPase, is highly useful to avoid a waste of PPase and the additionally required purification steps.

These and other problems are solved by the claimed subject matter, in particular by the employment of an immobilized PPase.

### SUMMARY OF THE INVENTION

As solution to the above discussed problems, the present invention provides a PPase immobilized onto a solid support, a method of producing the PPase and uses thereof. Further provided are an enzyme reactor and a kit comprising the PPase.

The immobilization of a PPase onto a solid support has a number of advantages over classical methods, wherein the PPase is free in solution together with the other components of the nucleic acid production reaction, such as RNA molecules, nucleotides, salts, buffer components etc.

First, a PPase which is immobilized onto a solid support may be used repeatedly and for the synthesis of different nucleic acid molecules which makes the reaction much more time-effective (quicker separation of the immobilized PPase), cost-effective and more ecologic since less chemicals and other materials are needed for provision of PPase and its separation from the RNA or DNA and other reaction components. Immobilization may also enhance the stability of the enzyme PPase compared to the soluble PPase since aggregation and denaturation of the protein may be reduced. Moreover, the provision of an immobilized PPase enables that the reaction (e.g., RNA, DNA synthesis) can be performed in a continuous fed-batch mode which has procedural advantages (higher yields can be obtained).

Second, the immobilization of PPase facilitates purification of the RNA or DNA. In fact, the removal of the reaction mixture enables a simple separation of the immobilized PPase from the other reaction components, consequently, destructive separation steps such as heat denaturation, extraction and precipitation may be avoided. This also reduces impurities (e.g., denatured PPase proteins or fragments) in the produced nucleic acid molecules.

Finally, the enzyme reactor and kit comprising the immobilized PPase provides for the scale-up and automation of the nucleic acid molecule production in order to provide high yields of DNA and RNA molecules in a reproducible and quick way. For example, immobilized PPase may be used in automated nucleic acid reaction methods which employ a polymerase selected from the group consisting of DNA dependent DNA polymerase, RNA dependent DNA polymerase, DNA dependent RNA polymerase and RNA dependent RNA polymerase, more preferably of methods selected from the group consisting of polymerase chain reaction, reverse transcription, RNA in vitro transcription and sequencing of nucleic acid molecules. Automation of said reaction methods and the separation of the RNA or DNA products together with the renewed utilization of PPase thus provides for an ecologic and economic production of nucleic acid molecules.

Hence, immobilization of PPase overcomes a number of drawbacks of state of the art nucleic acid production methods.

In the context of the invention, an immobilization via at least one thiol group of said PPase, e.g., allowing for a bond between the PPase and a solid support which is selected from the group consisting of disulfide bond, thioester bond, and thioether, is preferred. This way of immobilization also avoids the employment of amino groups which are regularly present in the active center of PPases. Clearly, an immobilization via an amino acid which is present in the active center of a PPase will severely affect the biological activity of the enzyme. Since cysteine residues are in general not very frequent in amino acid sequence and even less frequently found in the active center of a protein, these residues are chosen for the attachment to the solid support.

For immobilization via a thiol group of the PPase, the solid support comprises a reactive group selected from the group consisting of thiol, haloacetyl, pyridyl disulfide, epoxy, maleimide and mixtures thereof; preferably the reactive group is selected from the group consisting of thiol, epoxy, maleimide and mixtures thereof. Suitable reactive groups to generate thioether linkages comprise epoxy activated supports, maleimide activated supports and haloacetyl activated supports (iodoacetyl, bromoacetyl). Immobilization via haloacetyl supports generates hydroiodic or hydrobromic acid as a toxic by-product. Therefore, this way of immobilization is essentially suitable for non-pharmaceutical RNA and DNA synthesis e.g. DNA sequencing or PCR. In the context of pharmaceutical DNA and RNA production, maleimide and epoxy supports are preferred, with epoxy supports being most preferred, since no toxic by-products are formed in the immobilization reaction.

The inventors surprisingly found that PPase immobilized on an epoxy-activated solid support could be achieved without activity loss. The reaction conditions were chosen in a way that stable thioether linkages between PPase and support were generated. Epoxy supports have the advantage that they provide for robust immobilization under different immobilization conditions with respect to pH, salt concentration and other agents, such as reducing agents. Also, a change of reaction conditions, such as a change in pH, is believed to be tolerated more easily.

In RNA in vitro transcription (IVT) reactions, dithiothreitol (DTT) (or mercaptoethanol etc.) is commonly added as a reducing agent, since the activity of e.g. the DNA dependent RNA Polymerase (e.g., T7 Polymerase) is strongly impeded in the absence of a reducing agent (Chamberlin and Ring (1973) Journal of Biological Chemistry, 248:235-2244). In addition, internal cysteine residues present in the RNA polymerase enzymes may aggregate via intermolecular disulphide bridges in the absence of a reducing agent, which would also reduce the effectivity of an RNA in vitro transcription reaction.

In embodiments, where other RNA polymerases are used for IVT that do not require DTT or other reducing agents for being active, the immobilization of PPase via disulfide bridges is sufficient (e.g., via thiol activated supports).

Therefore the present invention provides an inorganic pyrophosphatase (PPase) characterized in that the PPase is a microbial PPase and immobilized onto a solid support via at least one thiol group of said PPase. Preferably, the microbial PPase is a bacterial PPase, archaeal PPase or a yeast PPase. The bacterial PPase is preferably derived from a bacterium selected from the group consisting of Escherichia coli, Thermus aquaticus and Thermus thermophilus, more preferably the bacterial PPase is derived from E. coli.

In another preferred embodiment, the PPase is thermostable, i.e. a thermostable PPase.

Preferably, the PPase is immobilized onto the solid support via a covalent bond.

In a preferred embodiment, the solid support comprises a reactive group selected from the group consisting of thiol, haloacetyl, pyridyl disulfide, epoxy, maleimide and mixtures thereof, preferably the reactive group is selected from the group consisting of thiol, epoxy, maleimide and mixtures thereof, most preferably the solid support comprises an epoxy group. More preferably, the solid support comprises a member selected from the group consisting of sepharose, agarose, sephadex, silica, metal and magnetic beads, methacrylate beads, glass beads, silicon, polydimethyl-siloxane (PDMS), plastic materials, porous membranes, papers, alkoxysilane-based sol gels, polymethylacrylate, polyacrylamide, cellulose, monolithic supports, expanded-bed adsorbents, nanoparticles and combinations thereof, preferably the solid support comprises methacrylate beads. Even more preferably, the solid support is selected from the group consisting of thiol sepharose, thiopropyl sepharose, thiol-activated sephadex, thiol-activated agarose, silica-based thiol-activated matrix, silica-based thiol-activated magnetic beads, pyridyl disulfide-functionalized nanoparticles, maleimide-activated agarose, epoxy methacrylate beads and mixtures thereof, preferably the solid support is epoxy methacrylate beads.

In a preferred embodiment, the at least one thiol group of said PPase is the thiol group of at least one cysteine residue of said PPase. More preferably, the PPase is immobilized onto the solid support via a bond selected from the group consisting of a disulfide bond, a thioester bond, a thioether bond and combinations thereof, preferably a thioether bond.

The PPase optionally comprises an amino acid sequence being at least 80% identical to an amino acid sequence as depicted in any one of SEQ ID NOs: 1 to 21, preferably comprises an amino acid sequence being at least 80% identical to any one of SEQ ID NOs: 1 and 10 to 21, more preferably at least 80% identical to any one of SEQ ID NOs: 1, 13 and 16, and most preferably at least 80% identical to SEQ ID NO: 1.

The PPase optionally comprises an amino acid sequence being at least 90% identical to an amino acid sequence as depicted in any one of SEQ ID NOs: 1 to 21, preferably comprises an amino acid sequence being at least 90% identical to any one of SEQ ID NOs: 1 and 10 to 21, more preferably at least 90% identical to any one of SEQ ID NOs: 1, 13 and 16, and most preferably at least 90% identical to SEQ ID NO: 1.

The PPase optionally comprises an amino acid sequence being at least 95% identical to an amino acid sequence as depicted in any one of SEQ ID NOs: 1 to 21, preferably comprises an amino acid sequence being at least 95% identical to any one of SEQ ID NOs: 1 and 10 to 21, more preferably at least 95% identical to any one of SEQ ID NOs: 1, 13 and 16, and most preferably at least 95% identical to SEQ ID NO: 1.

In another embodiment of the present invention, the PPase is mutated, and preferably comprises at least one newly introduced cysteine residue compared to a native PPase. Alternatively, the PPase may comprise only one cysteine residue or is mutated to comprise only one cysteine residue. Preferably, the PPase comprises only one cysteine residue at the C-terminus of the PPase, optionally connected to the PPase via a linker, preferably an oligopeptide linker, such as a linker comprising glycine and serine.

Further provided is a method for producing a PPase being a microbial PPase and immobilized onto a solid support via at least one thiol group of said PPase, comprising a step of

a) contacting the PPase in a reaction buffer with a solid support under conditions suitable for immobilizing the PPase onto the solid support via at least one thiol group of the PPase.

Preferably, step a) comprises the formation of at least one disulfide bridge, thioester bond or thioether bond. More preferably, step a) comprises the formation of a covalent bond between at least one cysteine residue of the PPase and a thiol group, a haloacetyl group, an epoxy group, a pyridyl disulfide and/or a maleimide group of the solid support, even more preferably an epoxy group.

Optionally, in step a) the pH in the reaction buffer is in the range from 7 to 8, preferably at 7.5±0.2. Optionally, in step a) the reaction buffer comprises a buffering agent selected from the group consisting of phosphate buffer, Tris-HCl buffer, 4-(2-hydroxyethyl)-1-piperazineethanesulfonic acid (HEPES) and acetate buffer, preferably phosphate buffer or Tris-HCl, more preferably phosphate buffer.

In a preferred embodiment, the reaction buffer in step a) further comprises a salt, preferably a lyotropic salt, more preferably a salt of sodium or potassium, most preferably sodium sulfide or sodium chloride. The salt may be present in a concentration of at least 0.4 M, preferably at least 0.5 M.

The method may further comprise prior to step a) a step of

b) contacting the solid support with a solution comprising bovine serum albumin (BSA).

The method may further comprise prior to step a) and step b) a step of

c) expressing the PPase in a suitable expression host.

The method may further comprise after step c) and prior to step a) or step b) a step of

d) purifying the PPase from the expression host.

Preferably, the PPase is a bacterial PPase, an archaeal PPase or a yeast PPase, more preferably a bacterial PPase, most preferably derived from E. coli or a thermostable PPase.

Also provided is a PPase obtainable by the method as described above.

Further provided is the use of a PPase being immobilized onto a solid support for producing nucleic acid molecules. Preferably, the PPase is used in a method in which pyrophosphate is generated, more preferably the PPase is used in a method which employs a polymerase selected from the group consisting of DNA dependent DNA polymerase, RNA dependent DNA polymerase, DNA dependent RNA polymerase, RNA dependent RNA polymerase and combinations thereof, even more preferably the method is selected from the group consisting of polymerase chain reaction, reverse transcription, RNA in vitro transcription, sequencing of nucleic acid molecules and combinations thereof.

In a preferred embodiment, the used PPase is a microbial PPase, optionally the microbial PPase is a bacterial PPase, archaeal PPase or a yeast PPase. Preferably, the bacterial PPase is derived from a bacterium selected from the group consisting of Escherichia coli, Thermus aquaticus and Thermus thermophilus, preferably from Escherichia coli.

In another preferred embodiment, the used PPase is thermostable.

Preferably, the PPase is immobilized onto the solid support via a covalent bond.

Preferably, the solid support onto which the used PPase is immobilized comprises a reactive group selected from the group consisting of thiol, haloacetyl, pyridyl disulfide, epoxy, maleimide and a mixture thereof, more preferably the reactive group is selected from the group consisting of thiol, epoxy, maleimide and mixtures thereof, most preferably the reactive group is an epoxy group. The solid support may comprise a member selected from the group consisting of sepharose, agarose, sephadex, agarose, silica, magnetic beads, methacrylate beads, glass beads and nanoparticles, preferably methacrylate beads. Preferably, the solid support is selected from the group consisting of thiol sepharose, thiopropyl sepharose, thiol-activated sephadex, thiol-activated agarose, silica-based thiol-activated matrix, silica-based thiol-activated magnetic beads, pyridyl disulfide-functionalized nanoparticles, maleimide-activated agarose, epoxy methacrylate beads and mixtures thereof, preferably the solid support is epoxy methacrylate beads.

In a very preferred embodiment, the used PPase is immobilized onto a solid support via at least one thiol group of said PPase, preferably the thiol group of said PPase is the thiol group of at least one cysteine residue of said PPase. More preferably, the PPase is immobilized onto the solid support via a bond selected from the group consisting of a disulfide bond, a thioester bond, a thioether bond and combinations thereof.

Optionally, the used PPase comprises an amino acid sequence being at least 80% identical to an amino acid sequence as depicted in any one of SEQ ID NOs: 1 to 21, preferably at least 80% identical to any one of SEQ ID NOs: 1 and 10 to 21, more preferably at least 80% identical to any one of SEQ ID NOs: 1, 13 and 16, and most preferably at least 80% identical to SEQ ID NO: 1.

Optionally, the used PPase comprises an amino acid sequence being at least 90% identical to an amino acid sequence as depicted in any one of SEQ ID NOs: 1 to 21, preferably at least 90% identical to any one of SEQ ID NOs: 1 and 10 to 21, more preferably at least 90% identical to any one of SEQ ID NOs: 1, 13 and 16, and most preferably at least 90% identical to SEQ ID NO: 1.

Optionally, the used PPase comprises an amino acid sequence being at least 95% identical to an amino acid sequence as depicted in any one of SEQ ID NOs: 1 to 21, preferably at least 95% identical to any one of SEQ ID NOs: 1 and 10 to 21, more preferably at least 95% identical to any one of SEQ ID NOs: 1, 13 and 16, and most preferably at least 95% identical to SEQ ID NO: 1.

Optionally, the used PPase is mutated, and preferably comprises at least one newly introduced cysteine residue compared to a native PPase. Optionally, the used PPase comprises only one cysteine residue or is mutated to comprise only one cysteine residue, such as at the C-terminus as described above and below.

Preferably, the used PPase is the PPase as described herein above and below. The use may comprise a step of A) contacting the PPase with pyrophosphate under conditions suitable for catalyzing the conversion of pyrophosphate into phosphate ions.

Further provided is an enzyme reactor (1) comprising a PPase being covalently immobilized onto a solid support or comprising a PPase as described herein above and below.

The enzyme reactor (1) may further comprise
1) at least one reaction module (2) comprising the PPase,
2) one or more devices for measuring and/or adjusting at least one parameter selected from the group consisting of pH, salt concentration, magnesium concentration, phosphate concentration, temperature, pressure, flow velocity, RNA concentration and nucleotide concentration.

Preferably, the at least one reaction module (2) comprises a solid support comprising a reactive group selected from the group consisting of thiol, halo acetyl, pyridyl disulfide, epoxy, maleimide and mixtures thereof, more preferably the reactive group is selected from the group consisting of thiol, epoxy, maleimide and mixtures thereof.

The solid support optionally comprises a member selected from the group consisting of sepharose, agarose, sephadex, agarose, silica, magnetic beads, methacrylate beads, glass beads and nanoparticles. Preferably, the solid support is selected from the group consisting of thiol sepharose, thiopropyl sepharose, thiol-activated sephadex, thiol-activated agarose, silica-based thiol-activated matrix, silica-based thiol-activated magnetic beads, pyridyl disulfide-functionalized nanoparticles, maleimide-activated agarose, epoxy methacrylate beads and mixtures thereof.

Preferably, the enzyme reactor (1) is suitable for the use as described herein above and below.

In a preferred embodiment, the enzyme reactor (1) further comprises

i) a reaction module (2) for carrying out nucleic acid molecule production reactions;
ii) a capture module (3) for temporarily capturing the nucleic acid molecules; and
iii) a control module (4) for controlling the in-feed of components of a reaction mix into the reaction module (2), wherein
the reaction module (2) comprises a filtration membrane (21) for separating nucleic acid molecules from the reaction mix; and wherein
the control of the in-feed of components of the reaction mix by the control module (4) is based on the concentration of nucleic acid molecules separated by the filtration membrane (21).

The filtration membrane (21) may be an ultrafiltration membrane (21), preferably said filtration membrane (21) has a molecular weight cut-off in a range from 10 to 100 kDa, 10 to 75 kDa, 10 to 50 kDa, 10 to 25 kDa or 10 to 15 kDa, further preferably the filtration membrane has a molecular weight cut-off in a range of 10 to 50 kDa.

The filtration membrane (21) may be selected from the group consisting of regenerated cellulose, modified cellulose, polysulfone (PSU), polyethersulfone (PES), polyacrylonitrile (PAN), polymethylmethacrylate (PMMA), polyvinyl alcohol (PVA) and polyarylethersulfone (PAES).

Optionally, said reaction module (2) comprises a DNA or RNA template immobilized on a solid support as basis for nucleic acid transcription reaction.

Preferably, the capture module (3) comprises a resin to capture the produced nucleic acid molecules and to separate the produced nucleic acid molecules from other soluble components of the reaction mix. More preferably, said capture module (3) comprises means (31) for purifying the captured produced nucleic acid molecules.

Even more preferably, said capture module (3) comprises means (32) for eluting the captured produced nucleic acid molecules, preferably by means of an elution buffer.

In a preferred embodiment, the enzyme reactor (1) further comprises a reflux module (5) for returning the residual filtrated reaction mix to the reaction module (2) from the capture module (3) after capturing the produced nucleic acid molecules, more preferably the reflux module (5) for returning the residual filtrated reaction mix is a pump (51). Optionally, the reflux module (5) comprises at least one immobilized enzyme or resin to capture disruptive components.

In a very preferred embodiment, the enzyme reactor (1) further comprises a sensor unit (33) which may be present at the reaction module (2), if present, at the capture module (3), if present, at the control module (4) and/or, if present, at the reflux module (5) for the real-time measurement of the concentration of separated nucleic acid molecules, the concentration of nucleoside triphosphates, and/or further reaction parameters, such as pH-value, reactant concentration, temperature and/or salinity.

Preferably, said sensor unit (33) measures the concentration of separated nucleic acids by photometric analysis.

The enzyme reactor (1) may be suitable to operate in a semi-batch mode or in a continuous mode.

It is highly preferred that the enzyme reactor (1) is adapted to carry out the method as described herein above and below.

Further provided us a kit comprising

a PPase characterized in that the PPase is immobilized onto a solid support, preferably the PPase is the PPase as described herein above and below,
a DNA or RNA polymerase and
at least one buffer selected from the group consisting of a PPase reaction buffer, a DNA polymerase reaction buffer, a RNA polymerase reaction buffer and combinations thereof.

### BRIEF DESCRIPTION OF THE DRAWINGS

FIG. 1: FIG. 1 depicts immobilization procedures for inorganic pyrophosphatase (PPase). Inorganic pyrophosphatase (protein) may be coupled by passive physical forces (A), by affinity capture (B) or by covalent bond (C) to a suitable solid support (S). As solid support materials, a planar surface (elongated rectangle), and two different globular supports are exemplified (round circle and triangle), such as beads. (A) The coupling via physical adsorption (arrow) can occur on various, often random residues on a protein. Physical adsorption is based on weak physical intermolecular interactions including electrostatic, hydrophobic, van der Waals, and hydrogen bonding interactions. (B) The coupling via affinity, comprising bio-affinity, can occur on specified positions on a protein. Bio-affinity immobilization is based on strong interactions of two biomolecules, where one interacting partner is fused to the protein (black square), and the other interacting partner is coated on the respective support material (black circle). (C) The coupling via covalent bond (bar-bell) can occur via specific reactive residues on a protein, such as thiol groups, such as of cysteine residues. A covalent bond is a strong chemical bond. Reactive residues on the protein and reactive groups on the support material, as described herein, need to be present to form covalent bonds.

FIG. 2: Schematic representation of an enzyme reactor (1) for nucleic acid synthesis, comprising immobilized inorganic pyrophosphatase according to the present invention. Inorganic pyrophosphatase (“PPase”) is immobilized onto a solid support, in this case immobilized onto beads (B). The PPase catalyzes the conversion of pyrophosphate (“PPi”) into two phosphate (“Pi”) molecules (“Reaction”) in the reaction module (2). In said reaction module (2), the nucleic acid synthesis reaction may also take place. After the reaction occurred, the nucleic acid molecules (“na”) may be separated from the immobilized PPase in the enzyme reactor via a filtration membrane (21), such as an ultrafiltration membrane, which does not allow the passage of the PPase immobilized onto—in this exemplary case—beads. In this particular example, the enzyme reactor (1) furthermore comprises a capture module (3) for temporarily capturing the generated nucleic acid molecules which is connected to the reaction module (2) via an outlet (22). The control of the in-feed of components (e.g., dNTPs, NTPs) of the reaction mix is controlled by the control module (4), connected to the reaction module (2) via an inlet (42). The feed-in flow is generated by a pump (43), wherein the flow is controlled based on the concentration of nucleic acid molecules (e.g., RNA, DNA), and/or dNTPs and/or NTPs and/or buffer conditions, measured by a sensor unit (33) connected to the reaction module (2), control module (4) and/or the capture module (3).

FIG. 3: FIG. 3 depicts examples of different configurations for reaction modules and enzyme reactors containing immobilized inorganic pyrophosphatase. (A) Stirred-tank batch reactors, (B) Continuous (stirred-tank) batch reactors, (C) Stirred tank-ultrafiltration reactor, Different components of the reactor types are indicated: (2) reaction module/reactor vessel, (6) immobilized enzyme, (7) stirrer, (8) inlet, (9) outlet, (21) ultrafiltration device (diagonal line: ultrafiltration membrane), (10) feed tube for ultrafiltration device, (5) recirculation tube/reflux module, (12) substrate/buffer tank, (13) packed bed tank, containing enzymes. Figure adapted from (Illanes, Andrés, ed. Enzyme biocatalysis: principles and applications. Springer Science & Business Media, 2008).

FIG. 4: FIG. 4 shows the results of the colorimetric activity assay. The activity of PPase-beads is shown, expressed as units (“U”) PPase per μL. The buffers used for immobilization are indicated: 1 (100 mM Na₂HPO₄—HCl, pH 7.5, 500 mM NaCl); 2 (0.4 M Na₂SO₄, pH 7.5, 50 mM Na₂HPO₄); 3 (0.8 M Na₂SO₄, pH 7.5, 100 mM Na₂HPO₄). For a detailed description, see Example 1.

FIG. 5: FIG. 5 shows the results of the colorimetric activity assay. The activity of PPase-beads (“Beads”) compared to the activity of storage buffer supernatant (“SN”) without beads is shown, expressed as units PPase per μL. The buffers used for immobilization are indicated: 1 (100 mM Na₂HPO₄—HCl, pH 7.5, 500 mM NaCl); 2 (0.4 M Na₂SO₄, pH 7.5, 50 mM Na₂HPO₄); 3 (0.8 M Na₂SO₄, pH 7.5, 100 mM Na₂HPO₄). The activity of PPase-beads 3 weeks post immobilization is shown. For a detailed description, see Example 2.

### DEFINITIONS

For the sake of clarity and readability, the following definitions are provided. Any technical feature mentioned for these definitions may be read on each and every embodiment of the invention. Additional definitions and explanations may be specifically provided in the context of these embodiments. Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well-known and commonly employed in the art. Standard techniques are used for nucleic acid and peptide synthesis. The techniques and procedures are generally performed according to conventional methods in the art and various general references (e.g., Sambrook et al., 1989, Molecular Cloning: A Laboratory Manual, 2d ed. Cold Spring Harbor Laboratory Press, Cold Spring Harbor, N.Y.), which are provided throughout this document.

Enzyme: Enzymes are catalytically active biomolecules that perform biochemical reactions. One example of an enzyme is the inorganic pyrophosphatase (PPase) of the present invention which catalyzes the enzymatic conversion of PPᵢ into 2Pᵢ.

Nucleic acid producing enzymes: The term “nucleic acid producing enzymes” comprises virtually any enzyme that my produce a nucleic acid. Examples are DNA dependent DNA polymerase (e.g. Pol I-IV (prokaryotes); DNA-Polymerase α, β, γ, δ and ε (eukaryotes)), RNA dependent DNA polymerase (e.g., reverse transcriptase), DNA dependent RNA polymerase (e.g., phage T7, T3, SP6 Polymerases) and RNA dependent RNA polymerase (RdRp, RNA replicases of RNA viruses).

Protein: A protein typically comprises one or more peptides or polypeptides. A protein is typically folded into a 3-dimensional form, which may be required for the protein to exert its biological function. The sequence of a protein or peptide is typically understood to be the order, i.e. the succession of its amino acids. PPase is an exemplary protein.

Recombinant protein: The term “recombinant protein” refers to proteins produced in a heterologous system, that is, in an organism that naturally does not produce such a protein, or a variant of such a protein. In case, a protein is expressed from a typical expression vector in an expression host which also naturally expresses this protein—however—not in such increased quantities, such protein is also to be understood as “recombinant protein” in the sense of the present invention, e.g. native E. coli derived PPase expressed in E. coli as expression host. Typically, the expression systems used in the art to produce recombinant proteins are bacteria (e.g., Escherichia (E.) coli), yeast (e.g., Saccharomyces (S.) cerevisiae) or certain mammalian cell culture lines.

Expression host: An expression host denotes an organism which is used for recombinant protein production. General expression hosts are bacteria, such as E. coli, yeasts, such as Saccharomyces cerevisiae or Pichia pastoris, or also mammal cells, such as human cells.

PPase: PPase (inorganic pyrophosphatase) catalyzes the reaction PPᵢ->2Pᵢ. PPase has been widely used in methods wherein nucleic acid molecules are produced, such as RNA in vitro transcription reactions but also in DNA sequencing reactions and cDNA transcription reactions because the addition of PPase increases transcription yields and minimizes the effect of variation of magnesium concentration (see for example Cunningham P. R. and Ofengand J. (1990) Biotechniques 9(6): 713-714.). The addition of PPase to a reaction mixture (e.g., to RNA in vitro transcription reactions using DNA dependent RNA polymerases or to cDNA in vitro transcription using RNA dependent DNA polymerases) catalyzes the hydrolysis of inorganic pyrophosphate and thus prevents its direct inhibitory action of the transcription enzyme.

Nucleic acid molecules: The term “nucleic acid molecules” comprises deoxyribonucleic acid (DNA) molecules and ribonucleic acid (RNA) molecules. Also derivatives of DNA and RNA molecules may be encompassed by the term. Nucleic acid molecules are nucleotide polymers composed of nucleic acis monomers known as nucleotides. Each nucleotide has three components: a 5-carbon sugar, a phosphate group, and a nitrogenous base. If the sugar is deoxyribose, the polymer is DNA, if the sugar is ribose, the polymer is RNA.

RNA, mRNA: RNA is the usual abbreviation for ribonucleic acid. It is a nucleic acid molecule, i.e. a polymer consisting of nucleotides. These nucleotides are usually adenosine-monophosphate, uridine-monophosphate, guanosine-monophosphate and cytidine-monophosphate monomers which are connected to each other along a so-called backbone. The backbone is formed by phosphodiester bonds between the sugar, i.e. ribose, of a first and a phosphate moiety of a second, adjacent monomer. The specific succession of the monomers is called the RNA sequence. Usually, RNA may be obtainable by transcription of a DNA sequence, e.g., inside a cell. In eukaryotic cells, transcription is typically performed inside the nucleus or the mitochondria. In vivo, transcription of DNA usually results in the so-called premature RNA, which has to be processed into so-called messenger RNA, usually abbreviated as mRNA. Processing of the premature RNA, e.g. in eukaryotic organisms, comprises a variety of different posttranscriptional-modifications such as splicing, 5′-capping, polyadenylation, export from the nucleus or the mitochondria and the like. The sum of these processes is also called maturation of RNA. The mature messenger RNA usually provides the nucleotide sequence that may be translated into an amino acid sequence of a particular peptide or protein. Typically, a mature mRNA comprises a 5′-cap, a 5′-UTR, an open reading frame, a 3′-UTR and a poly(A) sequence.

In addition to messenger RNA, several non-coding types of RNA exist which may be involved in regulation of transcription and/or translation, and immunostimulation and which may also be produced by in vitro transcription. The term “RNA” further encompasses RNA molecules, such as viral RNA, retroviral RNA and replicon RNA, small interfering RNA (siRNA), antisense RNA, CRISPR/Cas9 guide RNA, ribozymes, aptamers, riboswitches, immunostimulating RNA (isRNA), transfer RNA (tRNA), ribosomal RNA (rRNA), small nuclear RNA (snRNA), small nucleolar RNA (snoRNA), microRNA (miRNA), and Piwi-interacting RNA (piRNA) etc.

DNA: DNA is the usual abbreviation for deoxyribonucleic acid. It is a nucleic acid molecule, i.e. a polymer consisting of nucleotide monomers. These nucleotides are usually deoxy-adenosine-monophosphate, deoxy-thymidine-monophosphate, deoxy-guanosine-monophosphate and deoxy-cytidine-monophosphate monomers which are—by themselves—composed of a sugar moiety (deoxyribose), a base moiety and a phosphate moiety, and polymerized by a characteristic backbone structure. The backbone structure is, typically, formed by phosphodiester bonds between the sugar moiety of the nucleotide, i.e. deoxyribose, of a first and a phosphate moiety of a second, adjacent monomer. The specific order of the monomers, i.e. the order of the bases linked to the sugar/phosphate-backbone, is called the DNA-sequence. DNA may be single-stranded or double-stranded. In the double stranded form, the nucleotides of the first strand typically hybridize with the nucleotides of the second strand, e.g. by A/T-base-pairing and G/C-base-pairing.

Sequence of a nucleic acid molecule/nucleic acid sequence: The sequence of a nucleic acid molecule is typically understood to be the particular and individual order, i.e. the succession of its nucleotides.

Sequence of amino acid molecules/amino acid sequence: The sequence of a protein or peptide is typically understood to be the order, i.e. the succession of its amino acids.

Sequence identity: Two or more sequences are identical if they exhibit the same length and order of nucleotides or amino acids. The percentage of identity typically describes the extent, to which two sequences are identical, i.e. it typically describes the percentage of nucleotides that correspond in their sequence position to identical nucleotides of a reference sequence. For the determination of the degree of identity, the sequences to be compared are considered to exhibit the same length, i.e. the length of the longest sequence of the sequences to be compared. This means that a first sequence consisting of 8 nucleotides/amino acids is 80% identical to a second sequence consisting of 10 nucleotides/amino acids comprising the first sequence. In other words, in the context of the present invention, identity of sequences preferably relates to the percentage of nucleotides/amino acids of a sequence, which have the same position in two or more sequences having the same length. Gaps are usually regarded as non-identical positions, irrespective of their actual position in an alignment.

The sequence identity may be determined using a series of programs, which are based on various algorithms, such as BLASTN, ScanProsite, the laser gene software, etc. As an alternative, the BLAST program package of the National Center for Biotechnology Information may be used with the default parameters. In addition, the program Sequencher (Gene Codes Corp., Ann Arbor, Mich., USA) using the “dirtydata”-algorithm for sequence comparisons may be employed.

The identity between two protein or nucleic acid sequences is defined as the identity calculated with the program needle in the version available in April 2011. Needle is part of the freely available program package EMBOSS, which can be downloaded from the corresponding website. The standard parameters used are gapopen 10.0 (“gap open penalty”), gapextend 0.5 (“gap extension penalty”), datafile EONAFULL (matrix) in the case of nucleic acids.

Vector: The term “vector” refers to a nucleic acid molecule, preferably to an artificial nucleic acid molecule. A vector in the context of the present invention is suitable for incorporating or harboring a desired nucleic acid sequence, such as a nucleic acid sequence comprising an open reading frame. Such vectors may be storage vectors, expression vectors, cloning vectors, transfer vectors etc. A storage vector is a vector, which allows the convenient storage of a nucleic acid molecule, for example, of an mRNA molecule. Thus, the vector may comprise a sequence corresponding, e.g., to a desired mRNA sequence or a part thereof, such as a sequence corresponding to the open reading frame and the 3′-UTR of an mRNA. An expression vector may be used for production of expression products such as RNA, e.g. mRNA, or peptides, polypeptides or proteins, such as the PPase of the present invention. For example, an expression vector may comprise sequences needed for transcription of a sequence stretch of the vector, such as a promoter sequence, e.g. an RNA polymerase promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector, which is suitable for transferring nucleic acid molecules into cells or organisms, for example, viral vectors. A vector in the context of the present invention may be, e.g., an RNA vector or a DNA vector. Preferably, a vector is a DNA molecule. Preferably, a vector in the sense of the present application comprises a cloning site, a selection marker, such as an antibiotic resistance factor, and a sequence suitable for multiplication of the vector, such as an origin of replication. Preferably, a vector in the context of the present application is a plasmid vector.

Immobilization: The term “immobilization” relates to the attachment of a molecule, in particular the PPase of the present invention, to an inert, insoluble material which is also called solid support.

Solid support: A solid support” is to be understood as any an inert, insoluble material which comprises at least one functional group suitable to form a bond with a functional group of a protein, such as PPase. Typical materials for solid supports are sepharose, agarose, sephadex, agarose, silica, magnetic beads, methacrylate beads, glass beads and nanoparticles. Solid supports may be beads or tubes, plates, grids and else.

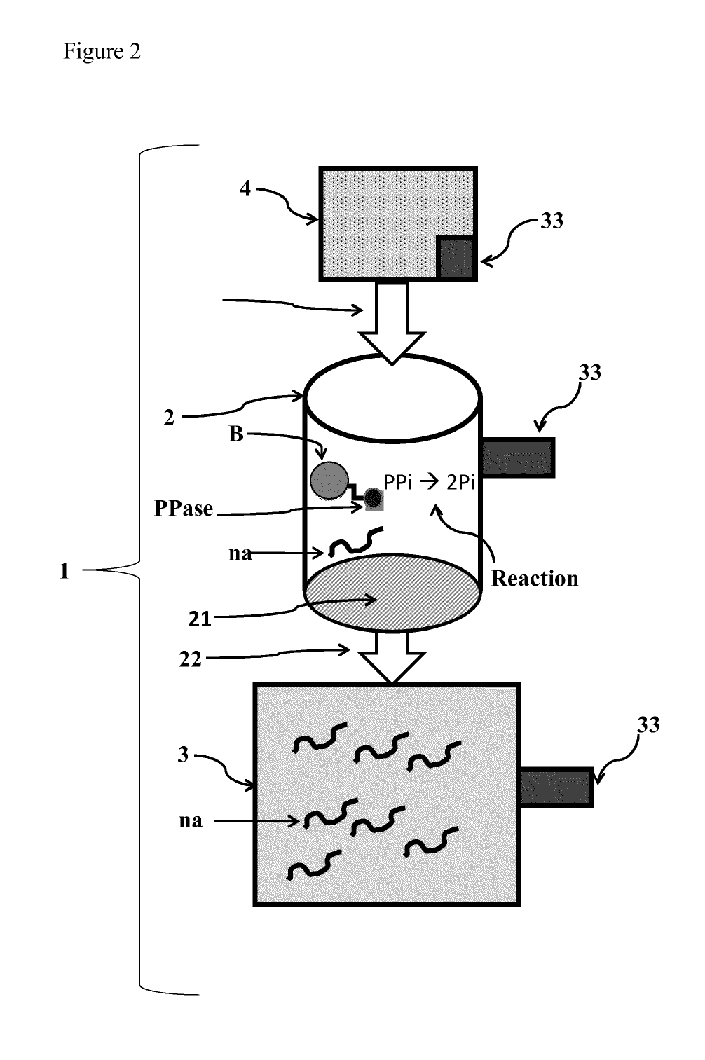

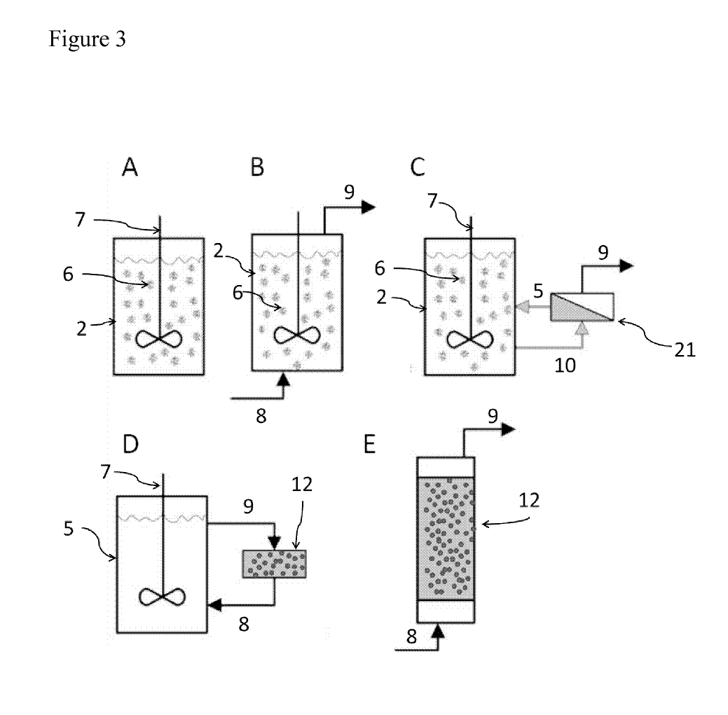

Enzyme reactor: An “enzyme reactor” also denoted as “bioreactor” may be any enzyme reactor comprising a vessel suitable for comprising the PPase of the present invention immobilized onto a solid support. The enzyme reactor is further suitable for comprising the other components of the PPase catalyzed reaction, such as PPᵢ, and components of methods for producing nucleic acid molecules, such as nucleotides, DNA dependent DNA polymerase, RNA dependent DNA polymerase, DNA dependent RNA polymerase and RNA dependent RNA polymerase, as well as water, buffer components and salts. That means the enzyme reactor is suitable so that the operator can apply the desired reaction conditions, e.g., temperature, reaction component concentration, salt and buffer concentration, pressure and pH value. The enzyme reactor further allows for the introduction and removal of the reaction components. An exemplary enzyme reactor is depicted in FIGS. 2 and 3.

Reaction components: “Reaction components” or “components of the PPase reaction” denote the components of the PPase catalyzed reaction, i.e. PPᵢ. Additional components are water, buffer components and salts. In the course of the reaction, phosphates emerging from the reaction PPᵢ->2Pᵢ are also considered to be reaction components.

Newly introduced amino acids: “Newly introduced amino acids” denote amino acids which are newly introduced into an amino acid sequence in comparison to a native amino acid sequence. Usually by mutagenesis, the native amino acid sequence is changed in order to have a certain amino acid side chain at a desired position within the amino acid sequence. In the present invention, in particular the amino acid cysteine is newly introduced into the amino acid sequence at one or more desired positions since the side chain of cysteine being a thiol group allows for easy and straightforward immobilization of the PPase onto a solid support via formation of a disulfide bridge, thioester bond or thioether bond, depending on the functional group of the solid support. The newly introduced amino acid may be introduced into the native or a mutated amino acid sequence between two amino acid residues already existing in the native or mutated amino acid sequence or may be introduced instead of an amino acid residue already existing in the native or mutated amino acid sequence, i.e. an existing amino acid is exchanged for the newly introduced amino acid sequence.

Functional group: The term is to be understood according to the skilled person's general understanding in the art and denotes a chemical moiety which is present on a molecule, in particular on the solid support, and which may participate in a covalent to another chemical molecule, such as PPase. Exemplary functional groups are thiol, haloacetyl, pyridyl disulfide, epoxy and a maleimide group.

Native amino acid sequence: The term is to be understood according to the skilled person's general understanding in the art and denotes the amino acid sequence in the form of its occurrence in nature without any mutation or amino acid amendment by man. Also called “wild-type sequence”. “Native PPase” denotes a PPase having the amino acid sequence as it occurs in nature. The presence or absence of an N-terminal methionine, which depends on the expression host used, usually does not change the status of a protein being considered as having its natural or native sequence.

Mutated: The term is to be understood according to the skilled person's general understanding in the art. An amino acid sequence is called “mutated” if it contains at least one additional, deleted or exchanged amino acid in its amino acid sequence in comparison to its natural or native amino acid sequence, i.e. if it contains an amino acid mutation. Mutated proteins are also called mutants. “Mutated to comprise only one cysteine residue” denotes that the amino acid sequence has been changed on the amino acid level so that the amino acid sequence contains only one cysteine residue. This may include that a cysteine residue was introduced via site-directed mutagenesis or one or more cysteine residues were removed, leaving only one cysteine residue in the amino acid sequence.

Microbial PPase: “Microbial PPase” denotes that the PPase is of microbial origin which includes bacterial PPase, archaeal PPase and yeast PPase.

Thermostable: “Thermostable” denotes that the PPase is able to properly catalyse the reaction PPᵢ->2Pᵢ at elevated temperatures, i.e. above 37° C., often above 50° C. Thermostable PPases are often derived from thermophilic bacteria and archaea, such as Thermus thermophilus, Thermus aquaticus and Thermococcus litoralis. Thermostable enzymes are of particular interest in polymerase chain reactions, wherein temperatures above 90° C. may be applied.

Reaction mix/reaction buffer: The terms “reaction mix” or “reaction buffer” denote a composition which provides a suitable chemical environment for a desired enzymatic reaction to take place. Hence, usually, a reaction mix or reaction buffer is an aqueous solution containing a buffering agent, such as phosphate buffer, acetate buffer or else, salts, a specific pH and further excipients which enable an enzyme to catalyze the desired chemical reaction. A “PPase reaction buffer” or “PPase reaction mix” is an aqueous solution containing a buffering agent to ensure the desired pH and salt conditions so that the PPase is able to catalyze the reaction PPᵢ into 2Pᵢ. An exemplary PPase reaction buffer is 50 μL 500 mM Tris-HCl pH 9.0, 1 μL 1M MgCl₂ in water. Since the PPase is used in methods for producing nucleic acid molecules, the reaction conditions in the reaction buffer/mix also need to be suitable for other enzymes which are present in the same reaction module (2). An exemplary enzyme which may be present in the same reaction module (2) is a DNA or RNA polymerase. “RNA polymerase reaction buffer” and “DNA polymerase reaction buffers” are thus buffer mixtures which enable the respective enzyme to catalyze the respective native enzymatic reaction. Typical reaction mixtures are known in the art and can be obtained from various manufacturers. An exemplary reaction buffer/mix for RNA in vitro transcription comprises a buffering agent, such as HEPES, a polyamine, such as spermidine, a reducing agent, such as DTT, and an inorganic salt, such as MgCl₂, a mixture of all four nucleoside triphosphates (NTP), namely adenosine triphosphate (ATP), guanosine triphosphate (GTP), cytidine triphosphate (CTP), and uridine triphosphate (UTP), e.g. 80 mM HEPES, 2 mM spermidine, 40 mM DTT, 24 mM MgCl₂, 13.45 mM NTP mixture. It may further comprise 16.1 mM cap analog (e.g. m7G(5′)ppp(5′)G (m7G)).

Polymerase chain reaction (PCR): “Polymerase chain reaction”, abbreviated as “PCR” is a technique which synthesizes multiple copies of one or more fragments of DNA from a single or multiple target templates, i.e. DNA molecules. The original PCR method is based on the thermostable DNA polymerase enzyme from Thermus aquaticus (Taq polymerase), which synthesizes a complimentary strand of a given DNA strand, i.e. DNA sequence, in a mixture containing the four nucleotides cytosine, guanine, adenine and thymine and a pair of DNA primers, each primer being complementary to a terminus of the target DNA sequence. The reaction mixture is heated to separate the double helix DNA molecule into individual strands containing the target DNA sequence and then cooled to allow the primers to hybridize with their complimentary sequences on the two separate strands and the Taq polymerase to extend the primers into new complimentary strands. Repeated heating and cooling cycles multiply the target DNA exponentially, for each newly formed double helix separates to become two templates for further synthesis. To date, many variants of this general procedure are known and commonly used.

Reverse transcription (RT) or reverse transcription polymerase chain reaction (RT-PCR): Both terms describe a variant of the PCR reaction. The synthesis of DNA from an RNA template, i.e. an RNA molecule, via reverse transcription, produces the complementary DNA (cDNA) molecules. The enzymes reverse transcriptases (RTs) use an RNA template and a short primer complementary to the 3′ end of the RNA to direct the synthesis of the first strand cDNA, which can be used directly as a template for PCR. This combination of reverse transcription and PCR (RT-PCR) allows the detection of low abundance RNAs in a sample, and production of the corresponding cDNA, thereby facilitating the cloning of low copy genes. Alternatively, the first-strand cDNA can be made double-stranded using DNA Polymerase I and DNA Ligase. These reaction products can be used for direct cloning without amplification. In this case, RNase H activity, from either the RT or supplied exogenously, is required. See also Retroviruses, Coffin J. M., Hughes S. H., Varmus H. E., editors, Cold Spring Harbor Laboratory Press, 1997.

RNA in vitro transcription: “RNA In vitro transcription” is a method that allows for template-directed synthesis of RNA molecules of any sequence in a cell free system (in vitro). It is based on the engineering of a template that includes a bacteriophage promoter sequence (e.g. from the T7 coliphage) upstream of the sequence of interest followed by transcription using the corresponding RNA polymerase.

Particular examples of DNA-dependent RNA polymerases are the T7, T3, and SP6 RNA polymerases. A DNA template for RNA in vitro transcription may be obtained by cloning of a nucleic acid, in particular cDNA corresponding to the respective RNA to be in vitro transcribed, and introducing it into an appropriate vector for in vitro transcription, for example into plasmid DNA. In a preferred embodiment of the present invention, the DNA template is linearized with a suitable restriction enzyme, before it is transcribed in vitro. The cDNA may be obtained by reverse transcription of mRNA or chemical synthesis. Moreover, the DNA template for in vitro RNA synthesis may also be obtained by gene synthesis.

Methods for in vitro transcription are known in the art (Geall et al. (2013) Semin. Immunol. 25(2): 152-159; Brunelle et al. (2013) Methods Enzymol. 530:101-14). An exemplary reaction mix used in said method typically includes:

1) a linearized DNA template with a promoter sequence that has a high binding affinity for its respective RNA polymerase such as bacteriophage-encoded RNA polymerases;
2) ribonucleoside triphosphates (NTPs) for the four bases (adenine, cytosine, guanine and uracil);
3) optionally, a cap analog as defined below (e.g. m7G(5′)ppp(5′)G (m7G));
4) optionally, another modified nucleotide as defined below;
5) a DNA-dependent RNA polymerase capable of binding to the promoter sequence within the linearized DNA template (e.g. T7, T3 or SP6 RNA polymerase);
6) optionally a ribonuclease (RNase) inhibitor to inactivate any contaminating RNase;
7) a pyrophosphatase to degrade pyrophosphate, which inhibits transcription;
8) MgCl₂, which supplies Mg²⁺ ions as a co-factor for the polymerase;
9) a buffer to maintain a suitable pH value, which can also contain antioxidants (e.g. DTT), and/or polyamines such as spermidine at optimal concentrations, commonly based on Tris-HCl or HEPES.

In vitro transcribed RNA may be used in analytical techniques (e.g. hybridization analysis), structural studies (for NMR and X-ray crystallography), in biochemical and genetic studies (e.g. as antisense reagents), as functional molecules (ribozymes and aptamers) and in (genetic) vaccination, gene therapy and immunotherapy.

Modified nucleoside triphosphate: The term “modified nucleoside triphosphate” as used herein refers to chemical modifications comprising backbone modifications as well as sugar modifications or base modifications. These modified nucleoside triphosphates are also termed herein as (nucleotide) analogs, modified nucleosides/nucleotides or nucleotide/nucleoside modifications.

In this context, the modified nucleoside triphosphates as defined herein are nucleotide analogs/modifications, e.g. backbone modifications, sugar modifications or base modifications. A backbone modification in connection with the present invention is a modification, in which phosphates of the backbone of the nucleotides are chemically modified. A sugar modification in connection with the present invention is a chemical modification of the sugar of the nucleotides. Furthermore, a base modification in connection with the present invention is a chemical modification of the base moiety of the nucleotides. In this context nucleotide analogs or modifications are preferably selected from nucleotide analogs which are applicable for transcription and/or translation.

Sugar Modifications: The modified nucleosides and nucleotides, which may be used in the context of the present invention, can be modified in the sugar moiety. For example, the 2′ hydroxyl group (OH) can be modified or replaced with a number of different “oxy” or “deoxy” substituents. Examples of “oxy”-2′-hydroxyl group modifications include, but are not limited to, alkoxy or aryloxy (—OR, e.g., R═H, alkyl, cycloalkyl, aryl, aralkyl, heteroaryl or sugar); polyethyleneglycols (PEG), —O(CH₂CH₂ₒ)ₙCH₂CH₂OR; “locked” nucleic acids (LNA) in which the 2′ hydroxyl is connected, e.g., by a methylene bridge, to the 4′-carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy.

“Deoxy” modifications include hydrogen, amino (e.g. NH₂; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and 0.

The sugar group can also contain one or more carbons that possess the opposite stereochemical configuration than that of the corresponding carbon in ribose. Thus, a modified nucleotide can include nucleotides containing, for instance, arabinose as the sugar.

Backbone Modifications: The phosphate backbone may further be modified in the modified nucleosides and nucleotides. The phosphate groups of the backbone can be modified by replacing one or more of the oxygen atoms with a different substituent. Further, the modified nucleosides and nucleotides can include the full replacement of an unmodified phosphate moiety with a modified phosphate as described herein. Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

Base Modifications: The modified nucleosides and nucleotides, which may be used in the present invention, can further be modified in the nucleobase moiety. Examples of nucleobases found in RNA include, but are not limited to, adenine, guanine, cytosine and uracil. For example, the nucleosides and nucleotides described herein can be chemically modified on the major groove face. In some embodiments, the major groove chemical modifications can include an amino group, a thiol group, an alkyl group, or a halo group.

In particularly preferred embodiments of the present invention, the nucleotide analogs/modifications are selected from base modifications, which are preferably selected from 2-amino-6-chloropurineriboside-5′-triphosphate, 2-Aminopurine-riboside-5′-triphosphate; 2-aminoadenosine-5′-triphosphate, 2′-Amino-2′-deoxycyti-dine-triphosphate, 2-thiocytidine-5′-triphosphate, 2-thiouridine-5′-triphosphate, 2′-Fluorothymidine-5′-triphosphate, 2′-O-Methyl inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodo-cytidine-5′-triphosphate, 5-Iodo-2′-deoxycytidine-5′-triphosphate, 5-iodouridine-5′-triphosphate, 5-Iodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauri-dine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyl-adenosine-5′-triphosphate, N1-methylguanosine-5′-triphosphate, N6-methyladeno-sine-5′-triphosphate, O6-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deaza-guanosine-5′-triphosphate, 5-bromocytidine-5′-triphosphate, and pseudouridine-5′-triphosphate.

In some embodiments, modified nucleosides include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxy-methyl-pseudouridine, 5-propynyl-uridine, 1-propynyl-pseudouridine, 5-taurino-methyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudo-uridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In some embodiments, modified nucleosides include 5-aza-cytidine, pseudoisocytidine, 3-methyl-cytidine, N4-acetylcytidine, 5-formylcytidine, N4-methyl-cytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diamino-purine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methyl-thio-N6-threonyl carbamoyladenosine, N6,N6-dimethyladenosine, 7-methyladenine, 2-methylthio-adenine, and 2-methoxy-adenine.

In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methyl-guanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some embodiments, the nucleotide can be modified on the major groove face and can include replacing hydrogen on C-5 of uracil with a methyl group or a halo group. In specific embodiments, a modified nucleoside is 5′-0-(1-thiophosphate)-adenosine, 5′-0-(1-thiophosphate)-cytidine, 5′-0-(1-thiophosphate)-guanosine, 5′-0-(1-thiophos-phate)-uridine or 5′-0-(1-thiophosphate)-pseudouridine.

In further specific embodiments the modified nucleotides include nucleoside modifications selected from 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, 5-methyl-uridine, pyrrolo-cytidine, inosine, α-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, α-thio-adenosine, 8-azido-adenosine, 7-deaza-adenosine.

Further modified nucleotides have been described previously (see, e.g., WO 2013/052523).

5′-cap: A “5 ‘-cap” is an entity, typically a modified nucleotide entity, which generally “caps” the 5’-end of a mature mRNA. A 5′-cap may typically be formed by a modified nucleotide (cap analog, e.g., m7G(5′)ppp(5′)G (m7G)), particularly by a derivative of a guanine nucleotide. Preferably, the 5′-cap is linked to the 5′-terminus of a nucleic acid molecule, preferably an RNA, via a 5′-5′-triphosphate linkage. A 5′-cap may be methylated, e.g. m7GpppN (e.g. m7G(5′)ppp(5′)G (m7G)), wherein N is the terminal 5′ nucleotide of the nucleic acid carrying the 5′-cap, typically the 5′-end of an RNA. Such a 5′-cap structure is called cap0. In vivo, capping reactions are catalyzed by capping enzymes. In vitro, a 5′-cap may be formed by a modified nucleotide, particularly by a derivative of a guanine nucleotide. Preferably, the 5′-cap is linked to the 5′-terminus via a 5′-5′-triphosphate linkage.

A 5′-cap may be methylated, e.g. m7GpppN, wherein N is the terminal 5′ nucleotide of the nucleic acid carrying the 5′-cap, typically the 5′-end of an RNA. m7GpppN is the 5′-cap structure which naturally occurs in mRNA, typically referred to as cap0 structure.

Enzymes, such as cap-specific nucleoside 2′-O-methyltransferase enzyme create a canonical 5′-5′-triphosphate linkage between the 5′-terminal nucleotide of an mRNA and a guanine cap nucleotide wherein the cap guanine contains an N7 methylation and the 5′-terminal nucleotide of the mRNA contains a 2′-O-methyl. Such a structure is called the cap1 structure.

Further examples of 5′-cap structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5-dihydroxypentyl nucleotide, 3′-3 ‘-inverted nucleotide moiety, 3’-3′-inverted abasic moiety, 3′-2 ‘-inverted nucleotide moiety, 3’-2 ‘-inverted abasic moiety, 1,4-butanediol phosphate, 3’-phosphoramidate, hexylphosphate, aminohexyl phosphate, 3′-phosphate, 3′phosphorothioate, phosphorodithioate, or bridging or non-bridging methylphosphonate moiety. Further modified 5′-CAP structures which may be used in the context of the present invention are CAP1 (methylation of the ribose of the adjacent nucleotide of m7GpppN), CAP2 (methylation of the ribose of the 2nd nucleotide downstream of the m7GpppN), CAP3 (methylation of the ribose of the 3rd nucleotide downstream of the m7GpppN), CAP4 (methylation of the ribose of the 4th nucleotide downstream of the m7GpppN), ARCA (anti-reverse CAP analogue, modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

Purification: as used herein, the term “purification” or “purifying” is understood to mean that the desired nucleic acid in a sample is separated and/or isolated from impurities, intermediates, byproducts and/or reaction components present therein or that the impurities, intermediates, byproducts and/or reaction components are at least depleted from the sample comprising the nucleic acid. Non-limiting examples of undesired constituents of RNA-containing samples which therefore need to be depleted may comprise degraded fragments or fragments which have arisen as a result of premature termination of transcription, or also excessively long transcripts if plasmids are not completely linearized. Furthermore, intermediates may be depleted from the sample such as e.g. template DNA. Additionally, reaction components such as enzymes, proteins, bacterial DNA and RNA, small molecules such as spermidine, buffer components etc. may have to be depleted from the RNA sample. An example of a protein impurity present in a sample may be PPase. In addition, impurities such as, organic solvents, and nucleotides or other small molecules may be separated.

Sequencing of nucleic acid molecules: “Sequencing of nucleic acid molecules” denotes the determination of the specific order of nucleotides within a DNA molecule. It includes any method or technology used for determination of the order of the four bases, adenine, guanine, cytosine, and thymine, in a strand of DNA.

Gene therapy: Gene therapy may typically be understood to mean a treatment of a patient's body or isolated elements of a patient's body, for example isolated tissues/cells, by nucleic acids encoding a peptide or protein. It may typically comprise at least one of the steps of a) administration of a nucleic acid, preferably an artificial nucleic acid molecule as defined herein, directly to the patient—by whatever administration route—or in vitro to isolated cells/tissues of the patient, which results in transfection of the patient's cells either in vivo/ex vivo or in vitro; b) transcription and/or translation of the introduced nucleic acid molecule; and optionally c) re-administration of isolated, transfected cells to the patient, if the nucleic acid has not been administered directly to the patient.

(Genetic) vaccination: “Genetic vaccination” or “vaccination” may typically be understood to be vaccination by administration of a nucleic acid molecule encoding an antigen or an immunogen or fragments thereof. The nucleic acid molecule may be administered to a subject's body or to isolated cells of a subject. Upon transfection of certain cells of the body or upon transfection of the isolated cells, the antigen or immunogen may be expressed by those cells and subsequently presented to the immune system, eliciting an adaptive, i.e. antigen-specific immune response. Accordingly, genetic vaccination typically comprises at least one of the steps of a) administration of a nucleic acid, preferably an artificial nucleic acid molecule as defined herein, to a subject, preferably a patient, or to isolated cells of a subject, preferably a patient, which usually results in transfection of the subject's cells, either in vivo or in vitro; b) transcription and/or translation of the introduced nucleic acid molecule; and optionally c) re-administration of isolated, transfected cells to the subject, preferably the patient, if the nucleic acid has not been administered directly to the patient.

Immunotherapy: The term “immunotherapy” is to be understood according to the general understanding of the skilled person in the fields of medicine and therapy. Also used in this context are the terms “biologic therapy” or “biotherapy”. It is the treatment of a disease by inducing, enhancing, or suppressing an immune response in a patient's body and comprises in particular cancer immunotherapy. Immunotherapy is also being applied in many other disease areas, including allergy, rheumatoid disease, autoimmunity and transplantation, as well as in many infections, such as HIV/AIDS and hepatitis.

### DETAILED DESCRIPTION OF THE INVENTION

To solve the above mentioned problems, the present invention provides a PPase immobilized onto a solid support.

In a first aspect, the present invention provides a PPase characterized in that the PPase is a microbial PPase and immobilized onto a solid support via at least one thiol group of said PPase. The PPase is preferably a bacterial PPase, archaeal PPase or a yeast PPase, preferably a bacterial PPase. Further, the bacterial PPase may be derived from a bacterium selected from the group consisting of Escherichia coli, Thermus aquaticus and Thermus thermophilus. In a preferred embodiment of the present invention, the PPase is derived from E. coli. The use of microbial PPases has the additional advantage that they are often, such as in case of Escherichia coli (E. coli), commercially available and well characterized. Moreover, they can easily be recombinantly produced in standard expression hosts, such as in E. coli.

In a preferred embodiment the PPase is thermostable which makes it ideal for employment in polymerase chain reactions (PCR). Thermostable PPases are derived from thermophilic microorganisms and are able to operate at increased temperatures due to improved heat stability. Moreover, the improved heat stability may lead to a longer half-life/shelf-life of such immobilized enzymes.

Hence, thermostable PPases of bacteria from the bacteria order “Thermales” may be used in the context of the present invention, including bacterial PPases from the bacteria genus “Thermus”, “Meiothermus”, “Marinithermus”, “Oceanithermus” or “Vulcanithermus”.

In other embodiments, the PPase is an archaeal PPase. Respective PPases may be derived from an organism selected from the group consisting of Desulfurococcus, Staphylothermus marinus, Desulfurococcus, Staphylothermus hellenicus, Desulfurococcus fermentans, Pyrolobus fumarii, Thermogladius cellulolyticus, Thermosphaera aggregans, Sulfolobales archaeon, Thermosphaera aggregans, Thermofilum, Candidatus, Acidianus copahuensis, Sulfolobus acidocaldarius, Acidianus hospitalis, Metallosphaera sedula, Ignicoccus hospitalis, Ignicoccus islandicus, Thermofilum, Thermofilum pendens, Sulfolobus solfataricus, Pyrodictium occultum, Metallosphaera yellowstonensis, Hyperthermus butylicus, Pyrodictium delaneyi, Methanohalobium evestigatum, Pyrobaculum neutrophilum, Sulfolobus islandicus, Halococcus morrhuae, Pyrobaculum, Nitrososphaera viennensis, Haladaptatus cibariu, Aeropyrum camini, Candidatus nitrosopumilus, Candidatus Nitrosoarchaeum limnia, Methanosarcina, Nitrosopumilus, Methanobacterium sp., Nanoarchaeota archaeon 7A, Metallosphaera cuprina, Methanosalsum zhilinae, Halococcus thailandensis, Candidatus Nitrosopumilus salaria, Haladaptatus paucihalophilus, Candidatus Nitrosopumilus sp., Halolamina pelagica, Halogranum salarium, Halococcus sediminicola, Thermoproteus sp. AZ2, Haloferax sp. SB29, Halococcus hamelinensis, Methanosarcina sp. MTP4, Caldisphaera lagunensis, Methanosarcina barkeri 3, Natronomonas pharaonic, Methanosarcina flavescens, Caldivirga maquilingensis, Halorubrum kocurii and Halopiger djelfamassiliensis.

In a preferred embodiment of the present invention, the PPase is a recombinant PPase, i.e. a recombinantly produced PPase.

Preferably, the PPase of the present invention comprises an amino acid sequence being at least 50%, at least 55%, at least 60%, at least 65%, at least 70%, at least 75%, more preferably at least 80% or at least 85%, even more preferably at least 90%, or 95% or most preferably at least 98% or 99% identical to any of the amino acids depicted in SEQ ID NOs: 1 to 21 or to a native PPase sequence existing in nature.

The PPase may e.g. be selected from the group consisting of (UniProt database accession Nos. provided) P31414, AVP1_ARATH, Q56ZN6, AVP2_ARATH, Q9FWR2, AVPX_ARATH, Q06572, AVP_HORVU, P21616, AVP_VIGRR, Q8TJA9, HPPA1_C, Q8PYZ8, HPPA1_METMA, Q2RIS7, HPPA1_MOOTA, Q93AR8, HPPA1_MYCDI, Q93AS0, HPPA1_RHILT, Q8TJA8, HPPA2_METAC, Q8PYZ7, HPPA2_METMA, Q2RLE0, HPPA2_MOOTA, Q93AR9, HPPA2_MYCDI, Q93AS1, HPPA2_RHILT, Q8UG67, HPPA_AGRFC, Q8VNU8, HPPA_ALLVI, Q8VRZ1, HPPA_ANAMA, Q8A294, HPPA_BACTN, Q89K83, HPPA_BRADU, Q8YGH4, HPPA_BRUME, Q8G1E6, HPPA_BRUSU, Q8RCX1, HPPA_CALS4, Q3AFC6, HPPA_CARHZ, Q9A8J0, HPPA_CAUCR, Q8VNW3, HPPA_CHLAA, Q8KDT8, HPPA_CHLTE, Q898Q9, HPPA_CLOTE, Q8RHJ2, HPPA_FUSNN, Q8VNJ8, HPPA_HELCL, Q72Q29, HPPA_LEPIC, Q8F641, HPPA_LEPIN, Q93NB7, HPPA_MYXXA, Q82TF3, HPPA_NITEU, Q8VRZ2, HPPA_OCHA4, Q8ZWI8, HPPA_PYRAE, Q983A3, HPPA_RHILO, Q8VRZ3, HPPA_RHIME, Q8VPZO, HPPA_RHIRD, P60363, HPPA_RHOPA, Q8KY01, HPPA_RHOPL, O68460, HPPA_RHORT, Q82EJ8, HPPA_STRAW, Q9X913, HPPA_STRCO, Q9S5X0, HPPA_THEMA, Q8PH20, HPPA_XANAC, Q8P5M6, HPPA_XANCP Q93V56, IPYR1_ARATH, Q93Y52, IPYR1_CHLRE, Q889M7, IPYR1_PSESM, P21216, IPYR2_ARATH, Q949J1, IPYR2_CHLRE, Q9H2U2, IPYR2_HUMAN, Q91VM9, IPYR2_MOUSE, Q87WD6, IPYR2_PSESM, P87118, IPYR2_SCHPO, P28239, IPYR2_YEAST, O82793, IPYR3_ARATH, Q9LFF9, IPYR4_ARATH, O82597, IPYR5_ARATH, Q9LXC9, IPYR6_ARATH, Q9YBA5, IPYR_AERPE, Q8UC37, IPYR_AGRFC, O67501, IPYR_AQUAE, Q8GQS5, IPYR_AQUPY, Q757J8, IPYR_ASHGO, Q9KCG7, IPYR_BACHD, P19514, IPYR_BACP3, P51064, IPYR_BARBK, P82992, IPYR_BLAVI, P37980, IPYR_BOVIN, Q89WYO, IPYR_BRADU, P65744, IPYR_BRUME, P65745, IPYR_BRUSU, P57190, IPYR_BUCAI, Q8KA31, IPYR_BUCAP, P59417, IPYR_BUCBP, Q18680, IPYR_CAEEL, Q9PHM9, IPYR_CAMJE, P83777, IPYR_CANAL, Q6FRB7, IPYR_CANGA, Q9AC20, IPYR_CAUCR, Q821T4, IPYR_CHLCV, Q9PLF1, IPYR_CHLMU, Q9Z6Y8, IPYR_CHLPN, B0B8Z8, IPYR_CHLT2, Q3KKS0, IPYR_CHLTA, B0BAM7, IPYR_CHLTB, O84777, IPYR_CHLTR, Q8XIQ9, IPYR_CLOPE, Q8FMF8, IPYR_COREF, Q8NM79, IPYR_CORGL, P80887, IPYR_CYAPA, Q6BWA5, IPYR_DEBHA, P19371, IPYR_DESVH, Q54PV8, IPYR_DICDI, O77460, IPYR_DROME, P0A7B0, IPYR_ECO57, Q8FAG0, IPYR_ECOL6, P0A7A9, IPYR_ECOLI, Q5B912, IPYR_EMENI, Q8SR69, IPYR_ENCCU, P81987, IPYR_EUGGR, O05724, IPYR_GEOSE, O05545, IPYR_GLUOX, Q7VPC0, IPYR_HAEDU, P44529, IPYR_HAEIN, Q9HSF3, IPYR_HALSA, Q9ZLL5, IPYR_HELPJ, P56153, IPYR_HELPY, O23979, IPYR_HORVD, Q15181, IPYR_HUMAN, P13998, IPYR_KLULA, O34955, IPYR_LEGPN, Q6ACP7, IPYR_LEIXX, Q72MG4, IPYR_LEPIC, Q8EZ21, IPYR_LEPIN, Q4R543, IPYR_MACFA, O48556, IPYR_MAIZE, Q8TMI3, IPYR_METAC, Q8TVE2, IPYR_METKA, Q8PWY5, IPYR_METMA, O26363, IPYR_METTH, Q9D819, IPYR_MOUSE, P65747, IPYR_MYCBO, P47593, IPYR_MYCGE, O69540, IPYR_MYCLE, Q8EVW9, IPYR_MYCPE, P75250, IPYR_MYCPN, Q98Q96, IPYR_MYCPU, P9WI54, IPYR_MYCTO, P9WI55, IPYR_MYCTU, Q74MY6, IPYR_NANEQ, Q9JVG3, IPYR_NEIMA, Q9KOG4, IPYR_NEIMB, Q6MVH7, IPYR_NEUCR, P80562, IPYR_NOSS1, P81988, IPYR_OCHDN, A2X8Q3, IPYR_ORYSI, QODYB1, IPYR_ORYSJ, P57918, IPYR_PASMU, O13505, IPYR_PICPA, Q6KZB3, IPYR_PICTO, O77392, IPYR_PLAF7, Q5R8T6, IPYR_PONAB, Q9HWZ6, IPYR_PSEAE, P58733, IPYR_PSEAO, P0CAP8, IPYR_PSEGP, Q88QF6, IPYR_PSEPK, Q9UY24, IPYR_PYRAB, Q8U438, IPYR_PYRFU, O59570, IPYR_PYRHO, Q8XWX1, IPYR_RALSO, Q98ER2, IPYR_RHILO, Q92LH1, IPYR_RHIME, Q9RGQ1, IPYR_RHORT, Q1RIN6, IPYR_RICBR, Q92H74, IPYR_RICCN, Q4UKWO, IPYR_RICFE, Q9ZCW5, IPYR_RICPR, Q68WE9, IPYR_RICTY, P65749, IPYR_SALTI, P65748, IPYR_SALTY, P19117, IPYR_SCHPO, Q43187, IPYR_SOLTU, Q9X8I9, IPYR_STRCO, P50308, IPYR_SULAC, Q97W51, IPYR_SULSO, Q974Y8, IPYR_SULTO, P80507, IPYR_SYNY3, P37981, IPYR_THEAC, Q8DHR2, IPYR_THEEB, Q5JIY3, IPYR_THEKO, P77992, IPYR_THELN, P38576, IPYR_THET8, Q979E6, IPYR_THEVO, Q9PQH6, IPYR_UREPA, Q9KP34, IPYR_VIBCH, Q87SW1, IPYR_VIBPA, Q8DE89, IPYR_VIBVU, Q7MPD2, IPYR_VIBVY, Q8D274, IPYR_WIGBR, Q8PH18, IPYR_XANAC, Q8P5M4, IPYR_XANCP, P65750, IPYR_XYLFA, P65751, IPYR_XYLFT, Q6C1T4, IPYR_YARLI, P00817, IPYR_YEAST, Q8ZB98, IPYR_YERPE, Q9C0T9, IPYR_ZYGBA, QOVD18, LHPP_BOVIN, A5PLK2, LHPP_DANRE, Q9H008, LHPP_HUMAN, Q9D715, LHPP_MOUSE, Q5I0D5, LHPP_RAT, Q3B8E3, LHPP_XENLA, Q9X015, MAZG_THEMA, O29502, PPAC_ARCFU, C3PD52, PPAC_BACAA, C3LG25, PPAC_BACAC, Q81PH9, PPAC_BACAN, B7JRU3, PPAC_BACC0, Q736P6, PPAC_BACC1, B7IJP9, PPAC_BACC2, C1EXV7, PPAC_BACC3, B7HA46, PPAC_BACC4, B7HUD5, PPAC_BACC7, A7GQ01, PPAC_BACCN, B9J353, PPAC_BACCQ, Q81CE5, PPAC_BACCR, Q63AC7, PPAC_BACCZ, Q6HHR6, PPAC_BACHK, Q65E18, PPAC_BACLD, P56948, PPAC_BACME, A7ZAR2, PPAC_BACMF, A8FJD5, PPAC_BACP2, Q5WDX3, PPAC_BACSK, P37487, PPAC_BACSU, A9VIG8, PPAC_BACWK, C0ZGL2, PPAC_BREBN, Q97H75, PPAC_CLOAB, Q9RRB7, PPAC_DEIRA, Q834N3, PPAC_ENTFA, B1YLU2, PPAC_EXIS2, C4LOS9, PPAC_EXISA, Q5FK05, PPAC_LACAC, Q1GAB5, PPAC_LACDA, Q04AP3, PPAC_LACDB, Q043J4, PPAC_LACGA, A8YVH1, PPAC_LACH4, Q74JD5, PPAC_LACJO, Q9CEM5, PPAC_LACLA, Q88W32, PPAC_LACPL, Q92BR1, PPAC_LISIN, C1KV95, PPAC_LISMC, Q71ZM2, PPAC_LISMF, B8DE62, PPAC_LISMH, Q8Y757, PPAC_LISMO, A0AIQ0, PPAC_LISW6, Q58025, PPAC_METJA, A0BSR0, PPAC_METTP, Q8CUT9, PPAC_OCEIH, A7X451, PPAC_STAA1, A6U325, PPAC_STAA2, Q2FFH6, PPAC_STAA3, Q2FWY1, PPAC_STAA8, A5I U87, PPAC_STAA9, Q2YU53, PPAC_STAAB, Q5HEK1, PPAC_STAAC, P65752, PPAC_STAAM, P65753, PPAC_STAAN, Q6GFD7, PPAC_STAAR, Q6G813, PPAC_STARS, A8Z2T4, PPAC_START, P65754, PPAC_STAAW, B9DMR8, PPAC_STACT, Q5HN17, PPAC_STAEQ, Q8CNN7, PPAC_STAES, Q4L7N2, PPAC_STAHJ, Q49YW3, PPAC_STAS1, Q3K0B5, PPAC_STRA1, Q8E4D4, PPAC_STRA3, Q8DYS6, PPAC_STRA5, C0M7N7, PPAC_STRE4, B4U4N8, PPAC_STREM, P95765, PPAC_STRGC, O68579, PPAC_STRMU, P65757, PPAC_STRP1, Q04JL5, PPAC_STRP2, PODD14, PPAC_STRP3, B5E699, PPAC_STRP4, Q5XDN3, PPAC_STRP6, C1C8C3, PPAC_STRP7, P65759, PPAC_STRP8, A2RG81, PPAC_STRPG, B1ICV4, PPAC_STRPI, B8ZLD4, PPAC_STRPJ, P65755, PPAC_STRPN, PODD15, PPAC_STRPQ, B21R53, PPAC_STRPS, B5XJY9, PPAC_STRPZ, P65756, PPAC_STRR6, A4W395, PPAC_STRS2, COMGI9, PPAC_STRS7, A3CPM5, PPAC_STRSV, A4VWZ2, PPAC_STRSY, Q5M194, PPAC_STRT1, Q5M5T1, PPAC_STRT2, Q03M65, PPAC_STRTD, B9DTT7, PPAC_STRU0, C1CFB5, PPAC_STRZJ, C1CLN3, PPAC_STRZP, C1CSF2, PPAC_STRZT, Q9WZ56, PPAC_THEMA, B7GL49, PPAX_ANOFW, C3P0C8, PPAX_BACAA, C3LED0, PPAX_BACAC, AORKU8, PPAX_BACAH, Q6HQY9, PPAX_BACAN, B7JFI8, PPAX_BACC0, Q72XV8, PPAX_BACC1, B7IPS5, PPAX_BACC2, C1EZE2, PPAX_BACC3, B7HEG2, PPAX_BACC4, B7HWY7, PPAX_BACC7, B9J4R5, PPAX_BACCQ, Q81518, PPAX_BACCR, Q631J2, PPAX_BACCZ, Q9K6Y7, PPAX_BACHD, Q6HBC8, PPAX_BACHK, A7Z971, PPAX_BACMF, A8FHS1, PPAX_BACP2, Q9JMQ2, PPAX_BACSU, A9VQ75, PPAX_BACWK, Q8R821, PPAX_CALS4, Q8XIY6, PPAX_CLOPE, Q928B2, PPAX_LISIN, ClKYP8, PPAX_LISMC, Q71WU6, PPAX_LISMF, B8DBN0, PPAX_LISMH, Q8Y4G3, PPAX_LISMO, A0ALGS, PPAX_LISW6, Q8ENK3, PPAX_OCEIH, Q67YC0, PPSP1_ARATH, Q9FZ62, PPSP2_ARATH, Q9SU92, PPSP3_ARATH, Q5E9Y6, PRUNE BOVIN, Q86TP1, PRUNE_HUMAN, Q8BIW1, PRUNE_MOUSE, Q6AYG3, and PRUNE_RAT.

In essence, immobilization of the PPase can be performed in manifold ways, and may be applied in the context of the invention, exemplified in various reviews, including (Datta, Sumitra, L. Rene Christena, and Yamuna Rani Sriramulu Rajaram. 3 Biotech 3.1 (2013): 1-9.; Kim, Dohyun, and Amy E. Herr. Biomicrofluidics 7.4 (2013): 041501).

Inorganic pyrophosphatases have, besides other important structural features (e.g., mutimerization surfaces), binding pockets for substrates and active sites for the hydrolysis of pyrophosphate. All those key structural features have to be intact for proper enzyme functionality. Therefore, any coupling strategy should fulfill prerequisites for successful PPase immobilization as exemplified below.

(I) Enzymes should retain or enhance their biological activity after coupling.
(II) Immobilized enzymes should have similar or even a better long-term stability and thermal stability, leading to a longer shelf life.
(III) The sensitivity and reactivity of the enzyme should be preserved after immobilization.
(IV) The immobilization procedure should be strong enough and stable enough to minimize enzyme leakage or leakage of the support material or leakage of other chemicals involved in the immobilization process.

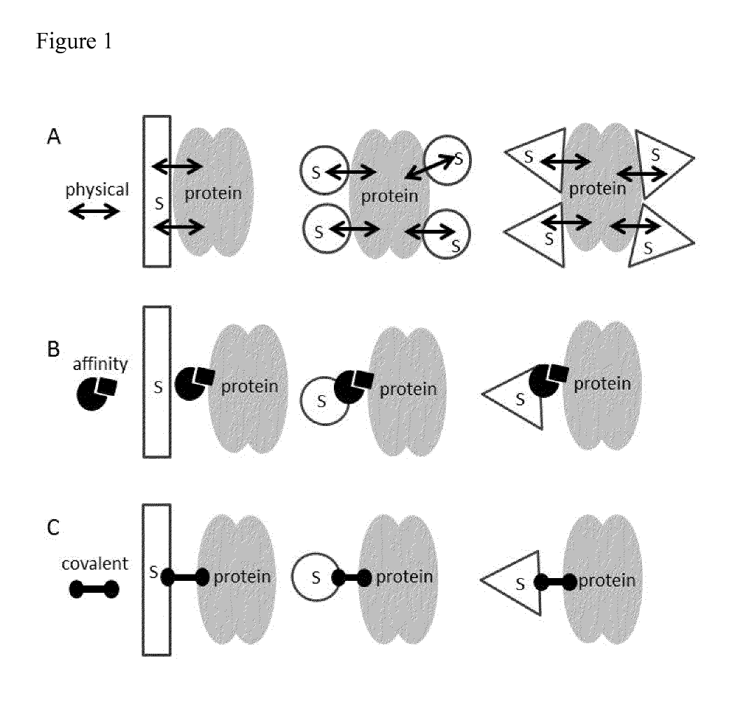

In principle, coupling strategies in the context of the invention mainly comprise, but are not limited to, entrapment/encapsulation, physical adsorption, bio-affinity interactions, and formation of a covalent bond. A schematic representation of possible immobilization strategies for PPase according to the present invention are shown in FIG. 1.

An immobilization support may comprise, but is not limited to, metals, silicon, glass, polydimethylsiloxane (PDMS), plastic materials, porous membranes, papers, alkoxysilane-based sol gels, agarose, sepharose, polymethylacrylate, polyacrylamide, cellulose, and silica, monolithic supports, and expanded-bed adsorbents. The choice of a suitable support material largely depends on the coupling strategy. Therefore potential support materials are mentioned in the context of the respective coupling strategy.

The basic principle of protein entrapment/encapsulation is that the respective enzyme may be encapsulated in the interior of the respective support material, which may prevent enzyme aggregation and enzyme denaturation.

Possible support materials comprise polyacrylamide gels, sol-gels, lipid vesicles and polymers such as poly (lactic acid) and poly (lactic-co-glycolic acid).

Physical adsorption, where the respective enzyme may bind passively on a particular support material, is based on physical forces such as electrostatic, hydrophobic, van der Waals, and hydrogen bonding interactions. Physical adsorption is based on random binding of the respective enzyme on multiple anchoring points to the support material.

Possible support materials comprise metal, silicon, glass, PDMS, and various adhesive plastic materials.

Bio-affinity immobilization strategies exploit the affinity interactions of different biological systems comprising the avidin-biotin system, and affinity capture ligands (His/GST tags).

In the widely employed avidin-biotin strategy, partners for biomolecules are avidin (tetrameric glycoprotein from chicken eggs), or neutravidin (deglycosylated version of avidin), or streptavidin (a protein form Streptomyces avidinii with higher affinity than avidin) and biotin (water soluble vitamin-B) that form strong non-covalent interactions. Biotinylated moieties strongly bind avidin or streptavidin. Biotinylation, that is the conjugation of biotin on molecules particularly proteins, does usually not affect functionality or conformation due to its small size. Inorganic PPase may be chemically or enzymatically biotinylated. Most chemical biotinylation reagents consist of a reactive group attached via a linker to the valeric acid side chain of biotin. As the biotin binding pocket in avidin or streptavidin is buried beneath the protein surface, biotinylation reagents possessing a longer linker are desirable, as they enable the biotin molecule to be more accessible to binding avidin or streptavidin protein. Chemical biotinylation may occur on several moieties in the respective enzyme including primary amines (—NH2), thiols (—SH, located on cysteines) and carboxyls (—COOH, a group located at the C-terminus of each polypeptide chain and in the side chains of aspartic acid and glutamic acid). All these above mentioned biotinylation targets in a protein can be used, depending on the respective buffer and pH conditions. For example, free thiol groups (sulfhydryl groups, —SH, located on cysteine side chains) are less prevalent on most proteins. Biotinylation of thiol groups is useful when primary amines are located in the regulatory domain(s) of the target protein or when a reduced level of biotinylation is required. Thiol-reactive groups such as maleimeides, haloacetyls and pyridyl disulfides require free thiol groups for conjugation; disulfide bonds must first be reduced to free up the thiol groups for biotinylation. If no free thiol groups are available, lysines can be modified with various thiolation reagents (Traut's Reagent, SAT (PEG4), SATA and SATP), resulting in the addition of a free sulfhydryl. Thiol biotinylation is performed in a pH range of 6.5-7.5.

Possible support materials for immobilizing inorganic PPase using the biotin-avidin strategy comprise, but are not limited to, agarose, sepharose, glass beads, which are coated with avidin or streptavidin. Particularly preferred is agarose and sepharose as support material.

Affinity capture ligands comprise, but are not limited to, oligohistidine-tag (His) and (glutathione-S-transferase) GST tags.

The C- or N-terminus of inorganic PPase may be genetically engineered to have a His segment (His tag) that specifically chelates with metal ions (e.g., Ni2 [symbol] ). Ni2 [symbol] is then bound to another chelating agent such as NTA (nitrilo acetic acid), which is typically covalently bound to an immobilization support material. The controlled orientation of respective enzyme may be facilitated, as the His tags can in principal be placed to the C- or N-terminus of each protein, and may be introduced at the C- or N-terminus of a PPase.

In addition, according to specific embodiments, a His segment as described above may be introduced for purification of a recombinant PPase according to the invention, e.g. in embodiments where the PPase is a recombinant protein produced in an expression host (e.g., E. coli).

Possible support materials comprise, but are not limited to, various nickel or cobalt chelated complexes, particularly preferred are nickel-chelated agarose or sepharose beads.

GST (glutathione S-transferase) may be tagged onto the C- or N-terminus (commonly the N-terminus is used) of the PPase by genetic engineering. The result would be a GST-tagged fusion protein. GST strongly binds to its substrate glutathione. Glutathione is a tripeptide (Glu-Cys-Gly) that is the specific substrate for glutathione S-transferase (GST). When reduced glutathione (G233SH) is immobilized through its thiol group to a solid support material, such as cross-linked beaded agarose or sepharose, it can be used to capture GST-tagged enzymes via the enzyme-substrate binding reaction.

Possible support materials comprise, but are not limited to, glutathione (GSH) functionalized support materials, particularly GSH-coated beads, particularly preferred GSH-coated agarose or sepharose.

Preferably, the PPase is immobilized onto the solid support by covalent binding.

Covalent immobilization is generally considered to have the advantage that the protein which is to be immobilized and the corresponding support material have the strongest binding, which is supposed to minimize the risk of proteins to dissociate from the support material, also referred to as enzyme leakage. Hence, covalent immobilization is preferred.

To achieve binding of the PPase to the support material, the respective support material has to be chemically activated via reactive reagents. Then, the activated support material reacts with functional groups on amino acid residues and side chains on the enzyme to form covalent bonds.

Functional groups on the PPase suitable for covalent binding comprise, but are not limited to, primary amines (—NH₂) existing at the N-terminus of each polypeptide chain and in the side-chain of lysine (Lys, K), α-carboxyl groups and the β- and γ-carboxyl groups of aspartic and glutamic acid, and sulfhydryl or thiol groups of cysteines. These functional groups are preferably located on the solvent exposed surface of the correctly 3 dimensionally folded PPase and preferably not located in the active center of the enzyme or in other key regions of the enzyme (as defined above).

Primary amines (—NH₂) provide a simple target for various immobilization strategies. This involves the use of chemical groups that react with primary amines. Primary amines are positively charged at physiologic pH; therefore, they occur predominantly on the outer surfaces of the protein, therefore, such groups are mostly accessible to immobilization procedures.

Suitable support materials for immobilization via primary amines comprise, but are not limited to, formaldehyde and glutaraldehyde activated support materials, 3-aminopropyltriethoxysilane (APTES) activated support materials, cyanogen bromide (CnBr) activated support materials, N-hydroxysuccinimide (NHS) esters and imidoesters activated support materials, azlactone activated support materials, and carbonyl diimidazole (CDI) activated support materials, epoxy activated support materials.

The carboxyl group is a frequent moiety (—COOH) at the C-terminus of each polypeptide chain and in the side chains of aspartic acid (Asp, D) and glutamic acid (Glu, E), usually located on the surface of protein structure. Carboxylic acids may be used to immobilize PPase through the use of a carbodiimide-mediated reaction. 1-ethyl-3(3-dimethylaminoipropyl) carbodiimide (EDC) and other carbodiimides cause direct conjugation of carboxylates (—COOH) to primary amines (—NH₂).

Possible support materials comprise, but are not limited to, diaminodipropylamine (DADPA) agarose resin that allow direct EDC-mediated crosslinking, which usually causes random polymerization of proteins.

The PPase of the invention is immobilized onto the solid support via at least one thiol group of the PPase which preferably forms a covalent bond with a functional group on the surface of the solid support. A covalent bond provides the strongest and most stable binding, which is supposed to minimize the risk of proteins to dissociate from the solid support, also referred to as enzyme leakage.

As outlined above, immobilization should consider that the enzyme needs to be accessible for the reaction substrates, i.e. the PPi molecules. Hence, it is beneficial to immobilize the PPase via an amino acid which is located on the surface of the protein when correctly folded into its 3-dimensional form and is not within the active center of the enzyme, i.e. not to an amino acid catalytically involved in the catalyzed reaction. This aspect is important so that the PPase retains its biological activity although immobilized onto a solid support.

In general, thiol groups are not found in the active center of PPases. Further, the small reactants PPᵢ and P of the reaction catalyzed by PPase allow for very flexible immobilization of PPase.

An immobilization support, i.e. the solid support of the invention, may comprise sepharose, agarose, sephadex, silica, metal and magnetic beads, methacrylate beads, glass beads, silicon, polydimethylsiloxane (PDMS), plastic materials, porous membranes, papers, alkoxysilane-based sol gels, polymethylacrylate, polyacrylamide, cellulose, monolithic supports, expanded-bed adsorbents, nanoparticles and combinations thereof.

The PPase of the invention is immobilized via a unique and mutually reactive group on the protein's surface, namely a thiol group, such as of the amino acid cysteine. Other options are amino acids which are chemically or enzymatically amended to possess a thiol group. Preferably, the immobilization is via a covalent bond. Alternatively, affinity binding or via physical attractive forces is also possible.

Many reactive groups used for covalent immobilization (see above) are commonly present multiple times in a protein. Due to the strong nature of covalent bonds, multiple bonds could, however, alter the 3-D conformation or destroy the catalytic core or other relevant protein domains. Therefore, complicated chemistry is often required to achieve oriented immobilization of enzymes (e.g., chemical blocking of other reactive groups in the enzyme such as ethanolamine to block excessive reactive amine groups). Site-specific covalent immobilization would allow the enzymes to be immobilized in a definite, oriented fashion. However, this process requires the presence of unique and mutually reactive groups on the protein (e.g., thiol group of cysteine) and the support (e.g., thiol activated sepharose). Furthermore, the reaction between the two reactive groups should be highly selective. Also, the coupling reaction should work efficiently under physiological conditions (i.e., in aqueous buffers around neutral pH) to avoid the denaturation of the protein during the immobilization step. Finally, it is desirable that the reactive group on the protein can be obtained using recombinant protein expression techniques.

Thiol groups, also called sulfhydryl groups, which have the structure R—SH, allow a selective immobilization of proteins and peptides as they commonly occur in lower frequencies (Hansen et al. (2009) Proc. Natl. Acad. Sci. USA 106.2: 422-427). Thiol groups may be used for direct immobilization reactions of PPase to activated solid support materials, forming e.g. thioether linkages (R—S—R) prepared by the alkylation of thiols or disulfide bonds (R—S—S—R) derived from coupling of two thiol groups or thioester linkages (thiolacid ester: R—C(O)—S—R, or thionacid ester: R—C(S)—O—R)). The thiol groups necessary for those reactions may have different sources:
a) Thiol groups of inherent or native free cysteine residues, in particular thiol groups which do not participate in disulfide bridges of the correctly 3-dimensionally folded protein.
b) Often, as part of a protein's secondary or tertiary structure, cysteine residues are joined together between their side chains via disulfide bonds. Thiol groups can be generated from existing disulfide bridges using reducing agents.
c) Thiol groups can be generated using thiolation reagents, which add thiol groups to primary amines.
d) Thiol groups can be genetically introduced by adding a cysteine residue at the C- or N-terminus or substituting an amino acid residue within the protein with another amino acid, particularly a cysteine. Thiol groups may also be introduced by introducing a cysteine residue into the natural amino acid sequence, preferably in a region of the protein which is neither important for the catalytic activity of the protein nor important for its structural integrity, such as often loop or turn structures.

Hence, the PPase is preferably immobilized onto the solid support via a bond selected from the group consisting of a disulfide bond, a thioester bond, a thioether bond and combinations thereof, more preferably a thioether bond.

The inventors consider this strategy to immobilize PPase via thiol groups of the protein to be generally advantageous because, commonly, only a low number of free existing thiol groups exist in the amino acid sequence of enzymes (Hansen et al. (2009) Proc. Natl. Acad. Sci. USA 106.2: 422-427).

This allows for a virtually site-specific and efficient way of immobilization. Such an oriented immobilization is preferred. Additionally, this immobilization strategy may avoid multiple coupling events to the solid support. Moreover, the covalent coupling via thiol groups of the PPase may have the advantage of a very strong bond that, most importantly, minimizes the danger of an uncontrolled dissociation of support material and enzyme, i.e. enzyme leakage.

Several different approaches exist in the art to bind a respective solid support to a thiol group of a protein. Thiol-reactive chemical groups present on support materials include maleimides, epoxy, haloacetyls, pyridyl disulfides and other disulfide reducing agents. Most of these groups conjugate to thiols on the respective protein by either alkylation (usually the formation of a thioether bond) or disulfide exchange (formation of a disulfide bond). The terms “functionalized” and “activated” with respect to the solid support are used interchangeable and refer to the chemical group which is available on the surface of the solid support for immobilization o the PPase.

For immobilization purposes via at least one thiol group of the PPase, the solid support preferably comprises a reactive group selected from the group consisting of thiol, haloacetyl, pyridyl disulfide, epoxy, maleimide and mixtures thereof, preferably the reactive group is selected from the group consisting of thiol, epoxy, maleimide and mixtures thereof, most preferably the reactive group is an epoxy group.

In a preferred embodiment, PPases are covalently coupled to the solid support via the thiol group of cysteine (native or introduced) to a support material, more preferably they are coupled via a disulfide bond to a thiol-activated solid support, via a thioether bond to a maleimide-activated solid support or to a pyridyl disulfide-functionalized solid support. Thiol-activated solid support contains chemical groups which are capable of reacting with the thiol group of the PPase, such as thiol, maleimides, epoxy, haloacetyls and pyridyl disulfides.

Maleimide-activated reagents react specifically with thiol groups (—SH) at near neutral conditions (pH 6.5-7.5) to form stable thioether linkages. The maleimide chemistry is the basis for most crosslinkers and labeling reagents designed for conjugation of thiol groups. Thiol-containing compounds, such as dithiothreitol (DTT) and beta-mercaptoethanol (BME), must be excluded from reaction buffers used with maleimides because they will compete for coupling sites.

Haloacetyls, such as iodoacetyl and bromoacetyl, react with thiol groups at physiological pH. The reaction of the iodoacetyl group proceeds by nucleophilic substitution of iodine with a sulfur atom from a thiol group, resulting in a stable thioether linkage. Using a slight excess of the iodoacetyl group over the number of thiol groups at pH 8.3 ensures thiol selectivity. Histidyl side chains and amino groups react in the unprotonated form with iodoacetyl groups above pH 5 and pH 7, respectively. To limit free iodine generation, which has the potential to react with tyrosine, histidine and tryptophan residues, iodoacetyl reactions and preparations should be performed in the dark.

Pyridyl disulfides react with thiol groups over a broad pH range (the optimum is pH 4 to 5) to form disulfide bonds. During the reaction, a disulfide exchange occurs between the molecule's —SH group and the reagent's 2-pyridyldithiol group. As a result, pyridine-2-thione is released and can be measured spectrophotometrically (Aₘₐₓ=343 nm) to monitor the progress of the reaction. These reagents can be used as crosslinkers and to introduce thiol groups into proteins. The disulfide exchange can be performed at physiological pH, although the reaction rate is slower than in acidic conditions. Further information on pyridyl disulfide reactive groups can be taken from van der Vlies et al. (2010, Bioconjugate Chem., 21 (4), pp 653-662).

Another very potent solid support is an epoxy functionalized solid support. Epoxy comprises the functional group as depicted in Formula (I):

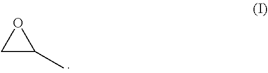

Epoxy-activated matrices can be used for coupling ligands stably through amino, thiol, phenolic or hydroxyl groups depending on the pH employed in the coupling reaction. Immobilization via epoxy groups is also described by Mateo et al., “Multifunctional epoxy supports: a new tool to improve the covalent immobilization of proteins. The promotion of physical adsorptions of proteins on the supports before their covalent linkage”, Biomacromolecules 1.4 (2000): 739-745. If the immobilization reaction takes place at a pH between 7.5-8.5, i.e. at physiological conditions, the attachment occurs at thiol groups, if the reaction takes place at a pH between 9 and 11, attachment occurs at amine residues and if the reaction takes place at a pH above 11, the attachment occurs at hydroxyl groups.

The solid support optionally comprises a member selected from the group consisting of sepharose, agarose, sephadex, silica, metal and magnetic beads, methacrylate beads, glass beads, silicon, polydimethylsiloxane (PDMS), plastic materials, porous membranes, papers, alkoxysilane-based sol gels, polymethylacrylate, polyacryl-amide, cellulose, monolithic supports, expanded-bed adsorbents, nanoparticles and combinations thereof.

Suitable solid supports include thiol sepharose, thiopropyl sepharose, thiol-activated sephadex, thiol-activated agarose, silica-based thiol-activated matrix, silica-based thiol-activated magnetic beads, pyridyl disulfide-functionalized nanoparticles, maleimide-activated agarose, epoxy methacrylate beads and mixtures thereof. Specific examples of thiol-activated sepharose are Thiol Sepharose 4B HiTrap or (Activated) Thiol Sepharose 4B or 6B (obtainable e.g. from GE, Fairfield, Conn., USA). Suitable pyridyl disulfide-functionalized supports include nanoparticles such as Nanosprings® of STREM chemicals or any amine-containing support thiolated by an N-Hydroxysuccinimide-pyridyl disulfide like NHS-PEG₄-pyridyl disulfide. Thiol-activated Sephadex G-10 (obtainable from GE, Fairfield, Conn., USA), thiol-activated agarose and maleimide-activated agarose may e.g. be obtained from Cube Biotech, Monheim am Rhein, Germany). Examples of Epoxy-activated resins are Purolite® ECR8205 epoxy methacrylate and Purolite® ECR8214 epoxy methacrylate are e.g. obtainable from Purolite® Corp., Llantrisant, UK, which are produced via crosslinking in the presence of a porogenic agent that allows the control of porosity, or ECR8204F epoxy-methacrylate beads which are obtainable from Lifetech™, Thermo Fisher Scientific, Waltham, Mass. USA). ECR8204F beads are of 150-300 μm diameter (mean=198) and pores of 300-600 Å. In further examples, the solid support comprises pyridyl disulfide-functionalized nanoparticles and/or maleimide-activated agarose.

The solid support may be a mixture of the solid supports mentioned herein. However, it is preferred to have the same functional group presented on the solid support, i.e. the thiol group. For example, in one single enzyme reactor thiol sepharose, thiopropyl-sepharose and thiol-activated sephadex may be used for immobilization of the PPase.

In embodiments where the PPase is immobilized via thiol-activated supports to generate disulphide-bonds (R—S—S—R), immobilized PPase may be re-solubilized using reducing agents such as DTT or mercaptoethanol, or low pH to potentially re-use the support material.

In particularly preferred embodiments, the PPase is immobilized via epoxy, or maleimide activated supports to generate stable thioether linkages (R—S—R). In said embodiments, the PPase is irreversible coupled to the support material. These embodiments are particularly preferred when the immobilized PPase is used in buffer conditions where reducing agents are required (e.g., DTT in buffers for RNA in vitro transcription).

Even more preferably, the PPase is immobilized via epoxy activated supports, particularly via epoxy methacrylate beads.

If a PPase is coupled via the thiol group of a cysteine to the solid support, several aspects should be considered by a person skilled in the art:

I) If several cysteine residues are present in the primary protein structure, free thiol groups, meaning cysteine residues not linked to other cysteine residues via disulfide bridges, may be identified using disulfide bridge prediction algorithms (Yaseen, Ashraf, and Yaohang Li. BMC bioinformatics 14. Suppl 13 (2013): S9.).
II) The freely existing thiol groups should not be present in the catalytic core or other functionally or structurally relevant parts of the PPASE/PAP since this would lower or could even destroy the enzymatic activity of the enzyme. Optionally, a person skilled in the art may first conduct the present literature on the structure of PPase or literature on structure-function relationships to identify such potential cysteine residues.
III) If several free thiol groups are present in the primary sequence of the protein, that are not located in the catalytic core or other functionally or structurally relevant parts of the PPase, respective cysteines may be substituted with a different amino acid, preferably serine (similar size) or alanine (similar charge), preferably by genetic means. This may help to avoid multiple coupling events to the solid support although, as mentioned above, PPase is highly tolerable to multi-site-immobilization. Protein visualization tools (e.g., PDB viewer, Guex and Peitsch (1997) Electrophoresis 18: 2714-2723) may help a person skilled in the art to decide whether respective cysteine residues should be substituted in the PPase. Alternatively, the skilled person may easily employ any of the immobilization strategies described herein and test the PPase for its catalytic activity. Moreover, the effect of certain cysteine substitutions and/or point mutations can also be estimated, even without structural knowledge, using machine-learning based prediction tools (Rost et al. (2004) Nucl. Acids Res. 32. suppl 2: W321-W326).
IV) If free thiol groups are present in the amino acid sequence of the respective PPase, a person skilled in the art may also use recent literature on the respective protein structure, if available, to assess if these cysteine residues are accessible for chemical interactions (i.e., covalent bond to a support material), or if these cysteine residues are buried in the interior of the protein's 3-D structure. A person skilled in the art may use algorithms to predict if a respective cysteine is buried or freely accessible by performing calculations comprising residue depth calculations or solvent-accessible surface area calculations (Xu, Dong, Hua Li, and Yang Zhang. Journal of Computational Biology 20.10 (2013): 805-816). Alternatively, the skilled person may easily employ any of the immobilization strategies described herein and test the PPase for its catalytic activity.
V) If no freely accessible cysteine residues are present in the primary structure of the respective PPase, cysteine residues may be introduced by various means. For example, cysteine residues may be introduced at the N-terminus or C-terminus of PPase by methods comprising genetic engineering, either by extending the N-terminus or the C-terminus or by substitution of the N-terminal-most or C-terminal-most amino acid. Moreover, a person skilled in the art may introduce flexible linkers, in particular, if the N- or C-terminus of the PPase displays important functional or structural features. Again, cysteine residues may also be introduced into any other suitable regions of the protein by substitution of amino acids within these regions. Ideally, such residues should be located on the protein surface and possibly in loop or turn structures which regularly do not play a role in the protein's structural integrity or are relevant for its enzymatic activity. Preferably, an amino acid that occupies a similar space in a protein's 3-D structure, such as serine, may be considered for an S to C substitution and vice versa if cysteine residues are to be removed. This will be explained in more detail below.

The cysteine residue preferably used for coupling may be present in the wild-type enzyme, i.e. in the natural amino acid sequence of PPase, if it is in a position suitable for coupling, or it may be introduced into the enzyme's amino acid sequence at a suitable position such as the N- or the C-terminus of the enzyme. The cysteine residue can be coupled to the N- or C-terminus directly, i.e. by forming a peptide bond with the N- or C-terminal amino acid of the wild-type PPase, or via a linker as defined herein. Alternatively, N- or C-terminal amino acid of the wild-type enzyme may be substituted with a cysteine residue.

Additionally, any cysteine residue present in the native/wild-type enzyme which is not suitable for coupling to a solid support may optionally be substituted with another amino acid, such as serine or alanine or valine, to avoid any residual coupling at this cysteine residue.

In a preferred embodiment, the PPase is immobilized via a thiol group which is present in the naturally occurring PPase. In another preferred embodiment, the PPase is immobilized via a newly introduced thiol group, i.e. via a newly introduced cysteine residue. Alternatively, the PPase is immobilized via a thiol group of a cysteine residue while one or more cysteine residues have been removed, i.e. have been replaced by alanine and/or serine residues. In another embodiment, the PPase is immobilized via a thiol group which is not present at a cysteine residue while one or more, or all cysteine residues have been removed from the amino acid sequence of the PPase.

The PPase may be mutated, and preferably the PPase is mutated to comprise at least one newly introduced cysteine residue compared to a native PPase. The PPase may also comprise only one cysteine residue or may be mutated to comprise only one cysteine residue.

In principle, introduction of cysteine residues in PPase is possible via the substitution of amino acids with cysteine at any position of the protein primary sequence or by extending the free N- or C-termini. However, several important aspects should be considered by a person skilled in the art if a cysteine residue is to be introduced into PPase via substitution:

I) Amino acids that are particularly important for the catalytic activity of PPase should not be substituted to cysteine.
II) Other amino acid residues that are located at the surface of PPase are potential targets for substitution with cysteine. Particularly, serine residues that have a similar size than cysteine residues may be preferred targets.
III) Amino acids that are not at the surface of PPase should not be changed to cysteine, as their thiol groups might not be in a position to react with the respective solid support. Moreover, a substitution of residues located in the interior of the protein may locally disrupt the protein structure.

Within the scope of the present invention not only the native PPase can be used, but also functional variants thereof. Functional variants of the PPase have a sequence which differs from that of the native PPase by one or more amino acid substitutions, deletions or additions, resulting in a sequence identity to the native PPase of at least 80%, preferably of at least 81%, 82%, 83%, 84% or 85%, more preferably of at least 86%, 87%, 88%, 89%, 90%, 91%, 92%, 93% or 94%, even more preferably of at least 95%, 96% and most preferably of at least 97%, 98% or 99%. Variants defined as above are functional variants, if they retain the biological function of the native and naturally occurring enzyme, i.e. the ability to catalyze the reaction PPᵢ->2Pᵢ. The enzyme activity of the functional variant of PPase is at least 50%, 60% or 70%, preferably at least 75%, 80% or 85%, more preferably at least 87%, 89%, 91% or 93% and most preferably at least 94%, 95%, 96%, 97%, 98% or 99% of the native enzyme as derivable from Escherichia coli (E. coli) (SEQ ID NO: 1), Thermus aquaticus (SEQ ID NO: 10) and Thermus thermophilus (SEQ ID NO: 15), preferably from E. coli as depicted in SEQ ID NO: 1.

Preferably, the PPase comprises an amino acid sequence being at least 80%, 85%, 90%, 95%, 98% or 99% identical to an amino acid sequence as depicted in SEQ ID NOs: 1 to 21. More preferably, the PPase comprises an amino acid sequence being at least 95% identical to an amino acid sequence as depicted in SEQ ID NOs: 1 (E. coli), 10 (Thermus aquaticus) and 15 (Thermus thermophilus)which are native PPase amino acid sequences. Alternatively, the PPase comprises an amino acid sequence being at least 95% identical to an amino acid sequence as depicted in SEQ ID NOs: 2 to 9, which are mutant sequences derived from SEQ ID NO: 1, to an amino acid sequence as depicted in SEQ ID NOs: 11 to 14, which are mutant sequences derived from SEQ ID NO: 10, or an amino acid sequence as depicted in SEQ ID NOs: 16 to 21, which are mutant sequences derived from SEQ ID NO: 15, wherein additional cysteine residues have been introduced to facilitate binding to a solid support or cysteine residues have been removed, e.g. by substitution with alanine residues to have a site-directed binding to the solid support. In SEQ ID NOs: 2 to 6, 9, 11, 12, 14 and 18 to 21, a linker, e.g. an amino acid sequence of glycine and serine residues has been attached to the C-terminus serving as a linker to a C-terminal cysteine. Examples of linkers with a C-terminal cysteine are -GGGGGC, -GGGGSGGGGC or -(GGGGS)₃C, Such linkers also facilitate binding of the enzyme to a solid support. When residues are to be introduced to serve as attachment point for immobilization onto a solid support, such as cysteine residues, this may be done at the N- or C-terminus or within the amino acid sequence.

Prior to amending the amino acid sequence, it is useful to use 3D structural data to evaluate whether the respective part of the sequence is relevant to the protein's structural integrity or plays a role in the enzymatic activity of PPase. The same applies if a residue is to be replaced or removed within the amino acid sequence. A residue, such as a cysteine residue, for immobilizing the enzyme to a solid support should be solvent exposed and not be relevant to the enzyme's structural integrity or biological function. It is further possible to identify the residues in the variant or mutant which correspond to those in the wild-type enzyme by aligning the amino acid sequences of the wild-type and variant enzymes using alignment software known to the skilled person.

The introduction or removal of cysteine residues is exemplified with E. coli PPase below. Several crucial residues have been determined to be important to the activity of the E. coli PPase (source: UniProtKB—P0A7A9 (IPYR_ECOLI) since the following mutations led to substantial activity loss: 21E→D: 16% activity; 30K→R: 2% activity; 32E→D: 6% activity; 44R→K: 10% activity; 52Y→F: 64% activity; 56Y→F: 7% activity; 66D→E: 6% activity; 68D→E: 1% activity; 71D→E: No activity; 98D→E: 22% activity; 98D→V: No activity; 99E→V: 33% activity; 103D→E: 3% activity; 103D→V: No activity; 105K→I: No activity; 105K→R: 3% activity; and 142Y→F: 22% activity. Hence, these amino acids are considered to be not suitable as immobilization point. However, the above list also implies that immobilization via internal cysteine residues (C54, C88) should not impair the catalytic activity of the enzyme.

To obtain a directed and controlled way of enzyme immobilization, one internal cysteine residue of the E. coli PPase may be replaced with another amino acid residue, preferably alanine, serine or valine, most preferably with an alanine and/or serine (e.g., C54A; C54S; C88A; C88S). The remaining cysteine residue in such mutated E. coli PPase may then be used for immobilization on an epoxy, haloacetyl, maleimide or thiol activated support.

In other embodiments, the E. coli PPase is immobilized via a newly introduced cysteine residue. In such an embodiment, all native cysteine residues, i.e. cysteine residues present in the native amino acid sequence of E. coli PPase, are replaced with another amino acid residue, preferably alanine, serine or valine, most preferably with an alanine and/or serine (C54A,C88A; C54S,C88S; C54A,C88S; C54S,C88A). Additionally, a cysteine residue is e.g. introduced at the N- or C-terminus of the protein. Said cysteine residues may be introduced e.g. by replacing the C-terminal lysine (K) with a Cysteine (K176C), which would e.g. result in a mutant enzyme C54A,C88A,K176C. In other embodiments, one or more cysteine residues within the amino acid sequence are removed and a new cysteine is added to the C-terminal end (e.g. C54A,C88A,177C, SEQ ID NO: 7), or by introducing the cysteine residue via a linker element (e.g., C54A,C88A,177GGGGGC, SEQ ID NO: 9). Said mutant enzymes may be immobilized using epoxy, maleimide, haloacetyl or thiol activated support materials (see above). The native and mutant amino acid sequences derived from E. coli are depicted in SEQ ID NOs: 1-9.

In other embodiments, the pyrophosphatase of the bacterium Thermus aquaticus is immobilized via an introduced cysteine residue (the wildtype or native enzyme does not have a cysteine) e.g. by adding a cysteine residue at the N- or C-terminus of the protein or elsewhere in the protein. Moreover, a cysteine residue may also be introduced e.g. by replacing the C-terminal arginine (R) with a cysteine (R175C). In other embodiments, a cysteine can be introduced by amending the protein sequence with a cysteine residue (e.g., 176C), or by introducing the cysteine residue via a linker element (e.g., 176GGGSGC). All mutant enzymes may be immobilized using epoxy, maleimide, haloacetyl or thiol activated support materials (see above). The native and mutant amino acid sequences derived from Thermus aquaticus are depicted in SEQ ID NOs: 10-14.

Various archaeal PPases do not harbor a native cysteine residue in the protein sequence. Such enzymes may be immobilized via introduced cysteines (N- or C-terminus or elsewhere in the amino acid sequence). For example, inorganic PPase from Staphylothermus marinus may be used, and a cysteine residue may be introduced by amending the sequence (177C); moreover, a cysteine residues may also be introduced e.g. by replacing the C-terminal methionine with a cysteine (M176C), or by introducing the cysteine residue with a linker element (e.g., 177linkerC). Said mutant enzymes may be immobilized using epoxy, haloacetyl, maleiimide or thiol activated support materials (as described elsewhere herein).

More preferably, the PPase of the invention comprises an amino acid sequence being at least 50%, at least 55%, at least 60%, at least 65%, at least 70%, at least 75%, more preferably at least 80% or at least 85%, even more preferably at least 90%, or 95% or most preferably at least 98% or 99% identical to any of the amino acid sequences depicted in SEQ ID NOs: 1 and 10 to 21, even more preferably as depicted in SEQ ID NOs: 1, 13 and 16 or to a native PPase sequence existing in nature (e.g. SEQ ID NOs: 1), most preferably as depicted in SEQ ID NO: 1.

Moreover, the flexible glycine/serine linker embodiments (e.g. in SEQ ID NOs: 2 to 6, 9, 11, 12, 14 and 18 to 21) can also be designed differently (only glycine, glycine-serine-linker, different amino acids, different linker length, etc.).

Accordingly, any other suitable linker may be used in the context of the invention (see for example Chen, Xiaoying, Jennica L. Zaro, and Wei-Chiang Shen (2013) Advanced drug delivery reviews 65.10:1357-1369).

In another aspect, methods are provided for producing the PPase of the present invention being a microbial PPase and immobilized onto a solid support via at least one thiol group of said PPase comprising a step of a) contacting the PPase with a solid support under conditions suitable for immobilizing the PPase onto the solid support via at least one thiol group of the PPase as explained above and as exemplified in the Examples section below. Preferably, the immobilization is via a covalent bond. More preferably, the immobilization in step a) leads to the formation of comprises the formation of at least one disulfide bridge, thioester bond or thioether bond. Specifically, it is preferred that step a) comprises the formation of a covalent bond between at least one cysteine residue of the PPase and a thiol group, a haloacetyl group, an epoxy group, a pyridyl disulfide and/or a maleimide group of the solid support. In another preferred embodiment, the solid support is a thiol-activated solid support, a haloacetyl functionalized solid support, pyridyl disulfide-functionalized solid support or epoxy-activated solid support or maleimide-activated solid support.

In an optional embodiment, in step a) of the method of the invention the pH in the reaction buffer is in the range from 5 to 9, preferably 7 to 8, and more preferably at 7.5±2.

The reaction buffer used in step a) may comprise a buffering agent as well known to the skilled person. Examples of buffering agents are phosphate buffer, Tris buffer, 4-(2-hydroxyethyl)-1-piperazineethanesulfonic acid (HEPES), acetate buffer and else. Preferably, the buffering agent is a Tris buffer or phosphate buffer, more preferably a phosphate buffer, e.g. Na₂HPO₄. The buffer in step a) may further comprise an inorganic salt, preferably a lyotropic salt, such as a sodium or potassium salt, more preferably, the buffer in step a), also denoted as “coupling buffer” or “immobilization buffer”, comprises sodium sulfate or sodium chloride. The inorganic salt may be present in a concentration of at least 0.3 mM, at least 0.4 M, at least 0.5, at least 7.5 M or more preferably at least 10 mM. Optionally, the reaction buffer in step a) may comprise EDTA.

In a preferred embodiment, in step a) the reaction buffer comprises

a1) 100 mM Na₂HPO₄—HCl and 500 mM NaCl at pH 7.5,
a2) 0.4 M Na₂SO₄ and 50 mM Na₂HPO₄ at pH 7.5,
a3) 0.8 M Na₂SO₄ and 100 mM Na₂HPO₄ at pH 7.5, or

### a4) 0.1 M Tris-HCl, 0.5 M NaCl, 1 mM EDTA at pH 7.5.

The method for producing the PPase may optionally comprise prior to step a) a step b) of contacting the solid support with a solution comprising bovine serum albumin (BSA). BSA serves as a filler material to occupy excessive reactive sites on the epoxy methacrylate beads and leads to a balanced distribution of immobilized enzymes per bead. In step b), the reaction conditions, in particular the reaction buffer is as in step a). BSA is e.g. used in a concentration of 20 mg/mL.

BSA may also be added to the reaction buffer in step a), optionally in a concentration of 20 mg/mL.

In the method for producing the PPase of the present invention, excessive reactive sites on the epoxy beads may be blocked using a cysteine solution, e.g. a 0.15 M cysteine solution which may be added to the reaction buffer at the end of step a). Incubation in step a) e.g. takes 4 h 50 min plus optional 15 min with cysteine solution. During incubation in step a), the reactions are rotated or stirred at approx. 12 rpm.

After step a), the PPase immobilized to the solid support is washed and stored in storage buffer. Exemplary washing is as follows: buffer 1: 1 mM MgCl2, 10 mM NaCl (Mg reconstitution of PPase); buffer 2: 10 mM Tris-HCl, pH8,0, 10 mM NaCl (low salt); buffer 3: 20 mM Tris-HCl, pH 8,0, 500 mM NaCl (high salt) and buffer 4: 20 mM Tris-HCl, pH8,0, 100 mM NaCl, 1 mM DTT, 0.1 mM EDTA (storage buffer). After adding the respective buffers, tubes were inverted and gently mixed for 10 seconds and centrifuged at at least 2000 rcf, e.g. 2340 rcf, for 1 minute, and the supernatant was removed. After the last washing step, storage buffer was added and the tubes were reverse-spinned at at least 2000 rcf, e.g. 2340 rcf, for 1 minute to move all the beads into the recovery cap. The obtained immobilized PPase may be stored in storage buffer at 5° C.

Optionally, the method for producing the PPase further comprises prior to step a) a step of b) expressing the PPase in a suitable expression host. The suitable expression host may be selected from a group consisting of a bacterial cell, a yeast cell or a mammalian cell. Preferably, the expression host is a bacterial cell, more preferably E. coli. Protein expression can be performed by standard methods well known to the skilled person such as described in Ceccarelli and Rosano “Recombinant protein expression in microbial systems”, Frontiers E-books, 2014, Merten “Recombinant Protein Production with Prokaryotic and Eukaryotic Cells. A Comparative View on Host Physiology”, Springer Science & Business Media, 2001, and others. There are also commercial suppliers who produce PPase on demand, such as Genscript, Piscataway, N.J., USA.

Optionally, the method of producing the PPase of the present invention further comprises after step b) and prior to step a) a step of c) purifying the PPase from the expression host. Protein purification may also be performed via standard procedures know to the skilled person. Further information can be obtained from Janson “Protein Purification: Principles, High Resolution Methods, and Applications”, John Wiley & Sons, 2012, and Burgess and Deutscher “Guide to Protein Purification”, Academic Press, 2009.

The produced PPase, preferably a bacterial PPase, an archaeal PPase or a yeast PPase, may be stored in lyophilized form or dissolved in a suitable storage buffer such as a buffer comprising 20 mM Tris-HCl, pH 8.0, 100 mM NaCl, 1 mM DTT, and 0.1 mM EDTA.

A preferred PPase of the present invention is a PPase produced by the above method.

The activity of the produced PPase may be tested using a colorimetric assay as described in detail in the Example section.

Further provided is the use of a PPase being immobilized onto a solid support for producing nucleic acid molecules. General information on these kind of methods can be taken from “Nucleic Acid Amplification Technologies: Application to Disease Diagnosis” (1997), Helen H. Lee, Springer Science & Business Media.

Preferably, the PPase is used in a method in which pyrophosphate is generated, more preferably the PPase is used in a method which employs a polymerase selected from the group consisting of DNA dependent DNA polymerase, RNA dependent DNA polymerase, DNA dependent RNA polymerase and RNA dependent RNA polymerase, even more preferably the method is selected from the group consisting of polymerase chain reaction, reverse transcription, RNA in vitro transcription and sequencing of nucleic acid molecules. Further information on DNA dependent DNA polymerases which produces DNA nucleic acid molecules from a single original DNA molecule can be gained from Kucera R. B. and Nichols N.M. (2008) Curr Protoc Mol Biol., Chapter 3, unit 3.5, John Wiley & Sons, Inc. and Knopf C. W. (1998) Virus Genes, 16(1):47-58. Further information on RNA dependent DNA polymerases which is a DNA polymerase enzyme that catalyzes the process of reverse transcription can be gained from Tzertzinis G., et al. (2008) Curr Protoc Mol Biol., Chapter 3, unit 3.7, John Wiley & Sons, Inc. Further information regarding DNA dependent RNA polymerase which catalyzes the synthesis of a complementary strand of RNA from a DNA template, or, in some viruses, from an RNA template, can be found in Sonntag K. C. and Darai G. (1995) Virus Genes, 11(2-3):271-84. Information on RNA dependent RNA polymerase which is an enzyme that catalyzes the replication of RNA from an RNA template can be found in Ahlquist (2002) Science, 296:1270. The nucleic acids produced in the method in which the PPase of the present invention is used may then be used in gene therapy, (genetic) vaccination or immunotherapy.

In a preferred embodiment of the use of the PPase described herein, the use comprises a step of A) contacting the PPase with pyrophosphate under conditions suitable for catalyzing the conversion of pyrophosphate into phosphate ions.

The PPase used herein may be a microbial PPase, preferably a bacterial PPase, archaeal PPase or a yeast PPase. The bacterial PPase is derived from a bacterium selected from the group consisting of Escherichia coli, Thermus aquaticus, and Thermus thermophilus. In a preferred embodiment, the PPase is thermostable. More preferably, the used PPase is immobilized onto the solid support via a covalent bond and may be immobilized onto a solid support as described herein above and as exemplified in the Example section.

The used PPase may comprises an amino acid sequence being at least 80% identical to an amino acid sequence as depicted in SEQ ID NO: 1 to 21, and preferably comprises an amino acid sequence being at least 80% identical to SEQ ID NO: 1 and 10 to 21, more preferably comprises an amino acid sequence being at least 80% identical to SEQ ID NOs: 1, 13 and 16, most preferably SEQ ID NO: 1. In an optional embodiment, the used PPase is mutated, and preferably comprises at least one newly introduced cysteine residue compared to a native PPase or the used PPase comprises only one cysteine residue or is mutated to comprise only one cysteine residue. Preferably, the used PPase is the PPase as described herein elsewhere.

Particularly preferred is the use of a PPase immobilized on a solid support via stable irreversible thioether (R—S—R) linkages (as described herein elsewhere) in RNA in vitro transcription reactions, wherein the reaction buffer may contain a reducing agent (DTT, mercaptoethanol etc..). Further preferred is the use of the PPase of the present invention in a method selected from the group consisting of polymerase chain reaction, reverse transcription, RNA in vitro transcription and sequencing of nucleic acid molecules.

An exemplary PPase reaction buffer, i.e. a buffer in which the PPase is capable of catalyzing the enzymatic reaction PPᵢ->2Pᵢ, is 50 μL 500 mM Tris-HCl pH 9.0, 1 μL 1M MgCl₂ in water. Since the PPase is used in methods for producing nucleic acid molecules, the reaction conditions in the reaction buffer/mix also need to be suitable for other enzymes which are present in the same reaction module (2). An exemplary enzyme which may be present in the same reaction module (2) is a DNA or RNA polymerase. An exemplary reaction buffer/mix for RNA in vitro transcription comprises a buffering agent, such as HEPES, a polyamine, such as spermidine, a reducing agent, such as DTT, and an inorganic salt, such as MgCl₂, a mixture of all four nucleoside triphosphates (NTP), namely adenosine triphosphate (ATP), guanosine triphosphate (GTP), cytidine triphosphate (CTP), and uridine triphosphate (UTP), e.g. 80 mM HEPES, 2 mM spermidine, 40 mM DTT, 24 mM MgCl₂, 13.45 mM NTP mixture. It may further comprise 16.1 mM cap analog (e.g. m7G(5′)ppp(5′)G (m7G)).

Further provided is an enzyme reactor (1) comprising a PPase being covalently immobilized onto a solid support or comprising a PPase as described herein. Preferably, the PPase is a microbial PPase and immobilized onto a solid support via at least one thiol group of the PPAse. An exemplary enzyme reactor (1) is depicted in FIG. 2.

Optionally, the enzyme reactor (1) further comprises

1) at least one reaction module (2) comprising the microbial PPase,
2) one or more devices for measuring and/or adjusting at least one parameter selected from the group consisting of pH, salt concentration, magnesium concentration, phosphate concentration, temperature, pressure, flow velocity, RNA concentration and nucleotide concentration.

Any enzyme reactor known to a skilled person or in the art may be used according to the present invention.

In general, an enzyme reactor (1) comprises one or more reaction modules (2) used to perform the desired enzymatic reaction, i.e. PPᵢ->2Pᵢ. Hence, the enzyme reactor may contain all reaction components necessary to perform this reaction, also denoted as the reaction mix. The reaction mix at least comprises the immobilized PPase and pyrophosphate. Since the pyrophosphate is generated in a method which produced a nucleic acid molecule, e.g. DNA or RNA, the reaction mix usually also comprises a further enzyme for nucleic acid production, such as a polymerase, which may or may not be immobilized as well, DNA or RNA molecules as template for the nucleic acid producing reaction, nucleotides and often a primer. Clearly, further enzymes, buffer components, salts etc. may also be present in the reaction mix depending on the specific nucleic acid production method as defined herein elsewhere. In the course of the reaction, the reaction mix also comprises Pᵢ and the produced nucleic acid molecules. In a particularly preferred embodiment, the PPase of the present invention is used in a method of RNA in vitro transcription which may further comprise (1) a linearized DNA template with a promoter sequence that has a high binding affinity for its respective RNA polymerase such as bacteriophage-encoded RNA polymerases, (2) ribonucleoside triphosphates (NTPs) for the four bases (adenine, cytosine, guanine and uracil), (3) optionally, a cap analog as defined below (e.g. m7G(5′)ppp(5′)G (m7G)), (4) optionally, another modified nucleotide as defined below, (5) a DNA-dependent RNA polymerase capable of binding to the promoter sequence within the linearized DNA template (e.g. T7, T3 or SP6 RNA polymerase), (6) optionally a ribonuclease (RNase) inhibitor to inactivate any contaminating RNase, (7) MgCl₂, which supplies Mg²⁺ ions as a co-factor for the polymerase, (9) a buffer to maintain a suitable pH value, which can also contain antioxidants (e.g. DTT), and/or polyamines such as spermidine at optimal concentrations, commonly based on Tris-HCl or HEPES

Important reactor types that may be used for the present invention comprise, but are not limited to, variants of stirred-tank batch reactors, continuous stirred-tank batch reactors, recirculation batch reactors, stirred tank-ultrafiltration reactors, and continuous packed-bed reactors (Illanes, Andrës, ed. Enzyme biocatalysis: principles and applications. Springer Science & Business Media, 2008, chapter 5), FIG. 3.

All reactor types may additionally have heating/cooling devices, pressure devices, and the stirred reactors may contain elements to control the stirring efficiency. Moreover, some reactors may be connected to a filtration setup, comprising e.g. an ultrafiltration device. The term bioreactor or enzyme reactor as used herein also refers to a chamber or test tube or column, wherein the methods for producing nucleic acid synthesis are carried out under specified conditions.

An enzyme reactor (of any kind), including tubes, vessels and other parts (sensors), for use in the present invention may be made of plastic, glass or steel, such as stainless steel according to European standard EN 10088, for example 1.43XX, 1.44XX, 1.45XX, or else. The material of the reaction vessel is also to be selected to have no binding of any of the reaction components to the walls of the vessel which may introduce a contamination to the following reaction. Further, the material should neither have any influence on the reaction itself, nor have a risk of leakage of hazardous chemicals (e.g., bisphenol A) or allergens (e.g., heavy metals). The material should also be selected to not be corrosive, such as stainless steel, or should in any way negatively influence the immobilization of the PPase of the invention.

Stirred-tank batch reactors (FIG. 3A) may consist of a tank or reaction module (2) containing a rotating stirrer. The vessel may be fitted with fixed baffles to improve the stirring efficiency in the reaction module (2). The reaction module (2) may be loaded with the immobilized PPase in a respective reaction buffer, and the other reaction components. In such a reaction module (2), the immobilized PPAse and other molecules have identical residence times. After the enzymatic reaction occurred, and after emptying of the batch reactor, the immobilized PPase, Pᵢ, the nucleic acid molecules and further enzymes have to be separated. This can be done e.g. by a filter device or membrane herein denoted as filtration membrane (21). Alternatively, the separation may be performed via centrifugation, and ideally the immobilized PPase may be recycled for another reaction cycle. The filtration membrane (21) allows for the direct separation of the immobilized PPase from the other reaction components so that the PPase may stay in the reaction module (2).

A stirred-tank batch reactor is particularly preferred in the context of the present invention. In this context, it is particularly preferred to use PPase immobilized to sepharose as solid support in the reaction vessel. In this context, it is particularly preferred to use PPase immobilized to epoxy methacrylate beads in the reaction vessel.

In another preferred embodiment, the enzyme reactor (1) comprising the immobilized PPase is a continuous stirred-tank batch reactor.

Continuous stirred-tank batch reactors (FIG. 3B) may be constructed similar to stirred-tank batch reactors (see above, cf. FIG. 3A) with the main difference that continuous in and out flow via inlet and outlet tubes may be applied. One feature of such a reactor type is that the immobilized PPase and the other components of the reaction mix, such as Pᵢ, do not have identical residence times in the reaction module (2). Reaction medium, composed of further enzymes (which produce PPᵢ) buffer, salts, nucleotides and RNA or DNA, may be pumped into the reaction module (2) via an inlet that may be located at the bottom of the tank, and reaction buffer containing the Pᵢ and further nucleic acid molecules produced in the reaction module (2) may be moved off via an outlet attached at the top. Optionally, the nucleotides and other reaction components are constantly and repeatedly fed into the reactor vessel to have a good distribution of the reaction components which are not immobilized, such as PPase. Inlet and outlet flow may be controlled by a pumping device in such a way that the enzymatic reaction can occur. Moreover, outlet tubes may have molecular weight cutoff filters to avoid contamination of the product by immobilized PPase or the immobilized PPase may be immobilized on a net or a honeycomb like solid structure inside the reaction vessel. One advantage of such an embodiment is that the immobilized PPase does not have to be separated from the other reaction components, such as the nucleic acid molecules or Pᵢ.

In another preferred embodiment, the enzyme reactor (1) containing an immobilized PPase is a stirred tank ultrafiltration reactor.

A stirred tank-ultrafiltration reactor (FIG. 3C) may be constructed similar to stirred-tank batch reactors (see above, cf. FIGS. 3A and 3B), with the major difference that a small ultrafiltration device is connected to the reaction module (2) where the separation of product Pᵢ and immobilized PPase takes place. This separation may be facilitated via an ultrafiltration or diafiltration device, filtration membrane (21). In ultrafiltration, the membranes comprise a discrete porous net-work. The mixed solution is pumped across the membrane, smaller molecules pass through the pores (Pᵢ, nucleic acid molecules) while larger molecules (immobilized PPase and further immobilized enzymes) are retained. Typical operating pressures for ultrafiltration are 1 to 10 bar. The retention properties of ultrafiltration membranes are expressed as molecular weight cutoff (MWCO). This value refers to the approximate molecular weight (MW) of a dilute globular solute (i.e., a typical protein) which is 90% retained by the membrane. However, a molecule's shape can have a direct effect on its retention by a membrane. For example, elongated molecules such as nucleic acid molecules may find their way through pores that will retain a globular species of the same molecular weight (Latulippel and Zydney (2011) Journal of Colloid and Interface Science. 357(2):548-553). Preferred in this context are cellulose membranes having nominal molecular weight cutoffs of 100 to 300 kDa.

Eventually, the immobilized PPase may be captured in the ultrafiltration device and returned back to the reaction chamber.

In another preferred embodiment the enzyme reactor (1) comprising the immobilized PPase of the present invention is a recirculation batch reactor.

Recirculation batch reactors (FIG. 3D) may comprise a first vessel, connected via inlet and outlet tubes to a second vessel. The first reaction module (2) is loaded with an immobilized or non-immobilized enzyme which produced the nucleic acid molecule and thereby also PPᵢ. One advantage of such an embodiment is that the immobilized PPase does not have to be separated from the other immobilized enzymes and produced nucleic acid molecules.

In another preferred embodiment, the enzyme reactor comprising an immobilized PPase is a continuous packed bed reactor.

Continuous packed bed reactors (FIG. 3E) may consist of a reaction module (2) comprising PPase immobilized to a solid support. The reaction module (2) may be densely packed, thereby forming a bed containing the PPase immobilized to a solid support as well as the nucleic acid producing enzyme immobilized to a solid support. One feature of such a reactor type is that the immobilized PPase and produced Pᵢ do not have identical residence times in the reactor. Reaction medium, composed of the reaction components including nucleotides, template DNA/RNA, may be pumped into the packed bed reactor via an inlet that may be located at the bottom of the tank, and reaction medium containing the Pᵢ and/or produced nucleic acid molecules product may be moved off via an outlet attached at the top of the tank. Inlet and outlet flow may be controlled by a pumping device in such a way that the enzymatic reaction can occur. Moreover, outlet tubes may have molecular weight cutoff filters (filtration membrane (21)) to avoid contamination of the product by immobilized PPase and/or immobilized nucleic acid producing enzyme. One advantage of such an embodiment is that the immobilized PPAse does not have to be separated from the other reaction components by other means.

In a preferred embodiment, the at least one reaction module (2) comprises a solid support comprising a reactive group selected from the group consisting of thiol, haloacetyl, pyridyl disulfide, epoxy, maleimide and mixtures thereof, preferably the reactive group is selected from the group consisting of thiol, epoxy, maleimide and mixtures thereof. Preferably, the solid support comprises a member selected from the group consisting of sepharose, agarose, sephadex, agarose, silica, magnetic beads, methacrylate beads, glass beads and nanoparticles. More preferably, the solid support is selected from the group consisting of thiol sepharose, thiopropyl sepharose, thiol-activated sephadex, thiol-activated agarose, silica-based thiol-activated matrix, silica-based thiol-activated magnetic beads, pyridyl disulfide-functionalized nanoparticles, maleimide-activated agarose, epoxy methacrylate beads and mixtures thereof.

In another preferred embodiment, the enzyme reactor (1) is suitable for the use described herein, namely for the use of a PPase being immobilized onto a solid support for producing nucleic acid molecules, e.g. for use in a method in which pyrophosphate is generated, preferably in a method which employs a polymerase selected from the group consisting of DNA dependent DNA polymerase, RNA dependent DNA polymerase, DNA dependent RNA polymerase and RNA dependent RNA polymerase, more preferably the method is selected from the group consisting of polymerase chain reaction, reverse transcription, RNA in vitro transcription and sequencing of nucleic acid molecules.

Optionally, the enzyme reactor (1) comprises

i) a reaction module (2) for carrying out nucleic acid molecule production reactions;
ii) a capture module (3) for temporarily capturing the nucleic acid molecules; and
iii) a control module (4) for controlling the in-feed of components of a reaction mix into the reaction module (2), wherein
the reaction module (2) comprises a filtration membrane (21) for separating nucleic acid molecules from the reaction mix; and wherein
the control of the in-feed of components of the reaction mix by the control module (4) is based on the concentration of nucleic acid molecules separated by the filtration membrane (21).

According to a preferred embodiment of the present invention, the enzyme reactor (1) comprises a control module (4). Data collection and analyses by the control module (4) allows the control of the integrated pump system (actuator) for repeated feeds of components of the reaction mix, e.g. buffer components or nucleotides. Tight controlling and regulation allows performing the nucleic acid molecule production method and thus the conversion of PPᵢ into 2Pᵢ under an optimal steady-state condition resulting in high product yield.

According to a further preferred embodiment of the present invention, the enzyme reactor (1) operates in a semi-batch mode or in a continuous mode. The term semi-batch as used herein refers to the operation of all nucleic acid production methods, such as the in vitro transcription reaction as a repetitive series of transcription reactions. For example, the reaction is allowed to proceed for a finite time at which point the product is removed, new reactants added, and the complete reaction repeated. The term continuous-flow as used herein refers to a reaction that is carried out continually in a bioreactor core with supplemental reactants constantly added through an input feed line and products constantly removed through an exit port. A continuous-flow reactor controls reagent delivery and product removal through controlled device flow rates, which is advantageous for reactions with reagent limitations and inhibitory products.

The filtration membrane (21) separates nucleotides and Pᵢ from the reaction mix which produces the nucleic acid molecule. The introduction of a filtration membrane in a flow system, for example an ultrafiltration membrane, is used for separation of high molecular weight components, such as e.g. immobilized or non-immobilized enzymes and/or polynucleotides, i.e. the produced nucleic acid molecules, from low molecular weight components, such as oligonucleotides having less than 25 nucleotides or Pᵢ.

Suitable filtration membranes may consist of various materials known to a person skilled in the art (van de Merbel, 1999. J. Chromatogr. A 856(1-2):55-82). For example, membranes may consist of regenerated or modified cellulose or of synthetic materials. The latter include polysulfone (PSU), polyacrylo-nitrile (PAN), polymethylmethacrylate (PMMA), mixtures of polyarylether-sulfones, polyvinyl-pyrrolidone and polyamide (Polyamix, RTM). For example, the polysulfones include polyethersulfone (poly(oxy-1,4-10 phenylsulfonyl-1,4-phenyl), abbreviated PES). In some exemplary embodiments, polyethersulfone may be utilized as a semipermeable membrane for the use according to the disclosure. In some cases PES membranes include increased hydrophilicity (and/or the improved wettability of the membrane with water) compared to PSU membranes. In some embodiments, the wettability of PES membranes can, for example, be further increased by the inclusion of the water-soluble polymer polyvinylpyrrolidone.

An important parameter that influences the flux of molecules across the filtration membrane is the pore size or pore-size distribution. A filtration membrane is usually characterized by its molecular weight cut-off (MWCO) value, i.e. a specific size limitation, which is defined as the molecular mass of the smallest compound, which is retained for more than 90%. For each application, a proper MWCO value needs to be selected so that high molecular weight compounds are sufficiently retained, but at the same time a rapid transport of the analyte is ensured. The filtration membrane (21) may be an ultrafiltration membrane (21), and preferably has a molecular weight cut-off in a range from 10 to 100 kDa, 10 to 75 kDa, 10 to 50 kDa, 10 to 25 kDa or 10 to 15 kDa, further preferably the filtration membrane has a molecular weight cut-off in a range of 10 to 50 kDa. Optionally, the filtration membrane (21) is selected from the group consisting of regenerated cellulose, modified cellulose, PES, PSU, PAN, PMMA, polyvinyl alcohol (PVA) and polyarylethersulfone (PAES).

The reaction module (2) preferably comprises a DNA or RNA template immobilized on a solid support as basis for nucleic acid transcription reaction.

The capture module (3) optionally comprises a resin, i.e. solid phase, to capture the produced nucleic acid molecules and to separate the produced nucleic acid molecules from other soluble components of the reaction mix. Optionally, the capture module (3) comprises means (31) for purifying the captured produced nucleic acid molecules and/or means (32) for eluting the captured produced nucleic acid molecules, preferably by means of an elution buffer.

In a preferred embodiment, the enzyme reactor (1) further comprises a reflux module (5) for returning the residual filtrated reaction mix to the reaction module (2) from the capture module (3) after capturing the produced nucleic acid molecules, preferably the reflux module (5) for returning the residual filtrated reaction mix is a pump (51). In an alternative embodiment, the reflux module (5) comprises at least one immobilized enzyme or resin to capture disruptive components. Hence, the immobilized PPase of the present invention may also be present in the reflux module (5) or in the capture module (3).

In a preferred embodiment, the enzyme reactor (1) further comprises a sensor unit (33) which may be present at the reaction module (2), capture module (3) and/or control module (4). The sensor unit (33) is suitable for the real-time measurement of the concentration of separated nucleic acid molecules, the concentration of nucleoside triphosphates, and/or further reaction parameters, such as pH-value, reactant concentration, in- and out-flow, temperature and/or salinity, optionally, the said sensor unit (33) measures, as a nucleic acid production parameter, the concentration of separated nucleic acids by photometric analysis.

The sensor unit (33) may measure further nucleic acid production reaction parameters in the filtrated reaction mix, preferably wherein the further nucleic acid production reaction parameters are pH-value and/or salinity.

According to some embodiments, the enzyme reactor (1), more specifically, the sensor unit (33) comprises at least one ion-selective electrode, preferably for measuring the concentration of one or more types of ions in a liquid comprised in at least one compartment of the enzyme reactor (1), wherein the ion is preferably selected from the group consisting of H⁺, Na⁺, K⁺, Mg²⁺, Ca2⁺, Cl⁻ and PO₄³⁻.

In the context of the present invention, the term “ion-selective electrode” relates to a transducer (e.g. a sensor) that converts the activity of a specific ion dissolved in a solution into an electrical potential, wherein the electrical potential may be measured, for instance, by using a volt meter or a pH meter. In particular, the term ‘ion-selective electrode’ as used herein comprises a system, which comprises or consists of a membrane having selective permeability, wherein the membrane typically separates two electrolytes. An ion-selective electrode as used herein typically comprises a sensing part, which preferably comprises a membrane having selective permeability and a reference electrode. The membrane is typically an ion-selective membrane, which is characterized by different permeabilities for different types of ions. Preferably, the at least one ion-selective electrode of the enzyme reactor (1) comprises a membrane selected from the group consisting of a glass membrane, a solid state membrane, a liquid based membrane, and a compound membrane.

In preferred embodiments, the at least one ion-selective electrode comprises or consists of a system comprising a membrane, preferably a membrane as described herein, more preferably an electrochemical membrane, having different permeabilities for different types of ions, wherein the membrane, preferably a membrane as described herein, more preferably an electrochemical membrane, preferably separates two electrolytes. In one embodiment, the membrane comprises or consists of a layer of a solid electrolyte or an electrolyte solution in a solvent immiscible with water. The membrane is preferably in contact with an electrolyte solution on one or both sides. In a preferred embodiment, the ion-selective electrode comprises an internal reference electrode. Such internal reference electrode may be replaced in some embodiments, for example by a metal contact or by an insulator and a semiconductor layer. An ion-selective electrode permits highly sensitive, rapid, exact and non-destructive measurement of ion activities or ion concentrations in different media. Apart from direct measurements of ion activities or ion concentrations they can serve, in particular by using a calibration curve, for continuous monitoring of concentration changes, as elements for control of dosage of agents or as very accurate indicator electrodes in potentiometric titrations.

In preferred embodiments, the enzyme reactor (1) comprises at least one ion-selective electrode, preferably as described herein, for measuring the concentration of one or more types of ions in at least one compartment of the enzyme reactor (1). For example, the at least one ion-selective electrode may be used to measure the concentration of one or more types of ions in a reaction module, a control module, a capture module or a reflux module (5) of the enzyme reactor (1). Of course, it is possible to have one or more sensor units and ion-selective electrodes at the enzyme reactor (1), i.e. one or more or each of the capture module (3), reaction module (2), control module (4) and/or reflux module (5). Preferably, the at least one ion-selective electrode is used for measuring the concentration of one or more types of ions in the reaction module, more preferably in the reaction core or in the filtration compartment. Furthermore, the at least one ion-selective electrode may be comprised in a sensor unit of the enzyme reactor (1), preferably as defined herein. The one or more ion-selective electrodes may be located in the enzyme reactor (1) itself, in the reaction module (2), reflux module (5), capture module (3) or control module (4) of the enzyme reactor (1) or outside of the enzyme reactor (1) (e.g. connected to the enzyme reactor by a bypass or tube). In the context of the present invention, the phrase ‘the enzyme reactor (1) comprises at least one ion-selective electrode’ may thus refer to a situation, where the at least one ion-selective electrode is a part of the enzyme reactor (1), or to a situation, where the at least one ion-selective electrode is a separate physical entity with respect to the enzyme reactor (1), but which is used in connection with the enzyme reactor (1).

Preferably, the at least one ion-selective electrode is connected to a potentiometer, preferably a multi-channel potentiometer (for instance, a CITSens Ion Potentiometer 6-channel, high-20 resolution; C-CIT Sensors AG, Switzerland). In a preferred embodiment, the at least one ion-selective electrode is preferably a tube electrode, more preferably selected from the group consisting of a Mg²⁺ selective tube electrode, a Na⁺ selective tube electrode, a Cl⁻ selective tube electrode, a PO₄³⁻ selective tube electrode, a pH-selective tube electrode and a Ca²⁺ selective tube electrode, preferably used in connection with a potentiometer. Even more preferably, the enzyme reactor (1) comprises at least one ion-selective electrode, wherein the at least one ion-selective electrode is preferably selected from the group consisting of a CITSens Ion Mg²⁺ selective mini-tube electrode, a CITSens Ion Na⁺ selective mini-tube electrode, a CITSens Ion CL selective mini-tube electrode, a CITSens Ion PO₄³⁻ selective mini-tube electrode, a CITSens Ion pH-selective mini-tube electrode and a CITSens Ion Ca²⁺ selective mini-tube electrode (all from C-CIT Sensors AG, Switzerland), preferably in connection with a potentiometer, more preferably with a multi-channel potentiometer, such as a CITSens Ion Potentiometer 6-channel, high-resolution (C-CIT Sensors AG, Switzerland).

Ion-selective electrodes have numerous advantages for practical use. For example, they do not affect the tested solution, thus allowing non-destructive measurements. Furthermore, ion-selective electrodes are mobile, suitable for direct determinations as well as titration sensors, and cost effective. The major advantage of the use of an ion-selective electrode in a enzyme reactor (1) (e.g. a transcription reactor) is the possibility to measure in situ without sample collection and in a non-destructive manner.

The ion-selective electrodes allow very specifically to monitor the nucleic acid production reaction, and in particular the reaction catalyzed by the immobilized PPase according to the invention.

The sensor unit (33) may further be equipped for the analysis of critical process parameters, such as pH-value, conductivity and nucleotide concentration in the reaction mix. Preferably, the sensor unit of the enzyme reactor (1) comprises a sensor, such as an UV flow cell for UV 260/280 nm, for the real-time measurement of the nucleotide concentration during the nucleic acid production method. Preferably, the sensor of the sensor unit measures the nucleotide concentration, as a process parameter, by photometric analysis.

The enzyme reactor (1) may operate in a semi-batch mode or in a continuous mode.

Moreover, the enzyme reactor (1) me be adapted to carry out the method as described herein and/or may comprise the PPase as described herein and/or may be suitable for the use described herein.

Further provided is a kit comprising a PPase characterized in that the PPase is immobilized onto a solid support, preferably the PPase is a microbial PPase or a PPase as described herein, a DNA or RNA polymerase and at least one buffer selected from the group consisting of a PPase reaction buffer, a DNA polymerase reaction buffer, a RNA polymerase reaction buffer and combinations thereof, including, e.g., nucleotides, salts etc.

In another aspect of the present invention, the produced nucleic acids according to the present invention may be used for the generation of genomic libraries or cDNA libraries.

In preferred embodiments of this aspect, the synthetized nucleic acids according to the present invention may be used in gene therapy, (genetic) vaccination or immunotherapy.

In a particularly preferred embodiment, the nucleic acid according to the invention is RNA, preferably in vitro transcribed RNA. Said RNA may then be used in gene therapy, (genetic) vaccination or immunotherapy.

### EXAMPLES

### Example 1: Immobilization of Inorganic Pyrophosphatase on Epoxy Methacrylate Beads

The goal of this experiment was the stable immobilization of inorganic pyrophosphatase (PPase). E. coli inorganic pyrophosphatase (SIGMA, SEQ ID NO: 1) was immobilized using ECR epoxy methacrylate beads (Lifetech™ ECR8204F). To obtain a balanced distribution of immobilized enzymes per bead, bovine serum albumin (BSA) was used as a filler material to occupy excessive reactive sites on the epoxy methacrylate beads. The reaction conditions, respectively the pH, were chosen as such the formation of thioether linkages (via sulfhydryl groups present on the PPase) was promoted. The obtained PPase-beads were tested for enzymatic activity and stability.

1. Reconstitution and Re-Buffering of E. coli PPase

1 mg (946 U) of inorganic E. coli PPase (SEQ ID NO: 1) was reconstituted in 2 mL water for injection. The dissolved protein was transferred to a Vivaspin-20 (30 kDa MWCO) column (Sartorius) and centrifuged for 15 minutes (5000 rpm; 22° C.). The flow through was discarded and 20 mL exchange buffer (50 mM Na₂HPO₄, pH 7.5, 50 mM NaCl) was added. After additional centrifugation for 20 minutes (5000 rpm; 22° C.), flow through was discarded and the retentate (approximately 200 μL) was stored at −25° C.

### 2. Immobilization Procedure

First sterile buffer solutions containing 20 mg/mL BSA were prepared (immobilization buffer 1: 100 mM Na₂HPO₄—HCl, pH 7.5, 500 mM NaCl; immobilization buffer 2: 0.4 M Na₂SO₄, pH 7.5, 50 mM Na₂HPO₄; immobilization buffer 3: 0.8 M Na₂SO₄, pH 7.5, 100 mM Na₂HPO₄). Second 0.5 g moist ECR epoxy methacrylate beads (Lifetech™ ECR8204F) were washed in centrifugation tubes (Vivaspin 2 VS0271 with 0.2 μm PES membranes), using 0.9 mL of the respective buffer without BSA, for 2 minutes at 2340 rcf (relative centrifugal force). After three washing steps, 2 mL of the respective BSA in buffer solutions was added. Then 30 μL of the re-buffered PPase solution (see above) was spiked into the reactions (buffer 1 and buffer 3) and 50 μL of re-buffered PPase solution was spiked into the reaction with buffer 2. The reactions were rotated for 4 hours and 50 minutes using a tube rotator at approximately 12 rpm (rotations per minute). Samples were taken after 40, 105, 160, 220 and 290 minutes. A sample of the BSA in buffer solutions was also taken as starting value. The samples were measured using a Qubit protein assay according to the manufacturer's instructions to assess the binding efficiency of PPase (and BSA) to the beads. To block excessive reactive sites on the epoxy beads, 400 μL of freshly prepared 0.15 M cysteine solution was added to each reaction and rotated for another 15 minutes. The tubes were then centrifuged at 2340 rcf for 1 minute.

The beads were washed using the following washing buffers (2 mL each) subsequently:
1 mM MgCl₂, 10 mM NaCl (Mg reconstitution of PPase)
10 mM Tris-HCl, pH 8.0, 10 mM NaCl (low salt)
20 mM Tris-HCl, pH 8.0, 500 mM NaCl (high salt)
20 mM Tris-HCl, pH 8.0, 100 mM NaCl, 1 mM DTT, 0.1 mM EDTA (storage buffer)

After adding the respective buffers, tubes were inverted and gently mixed for 10 seconds and centrifuged at 2340 rcf for 1 minute, and the supernatant was removed. After the last washing step, another 2 mL of storage buffer was added and the tubes were reverse-spinned at 2340 rcf for 1 minute to move all the beads into the recovery cap. The obtained PPase-beads were stored in storage buffer at 5° C.

3. Activity Tests of Immobilized E. coli PPase:

To test the obtained PPase-beads for its enzymatic activity, a colorimetric assay was performed. Moreover, respective supernatants (storage buffer alone without PPase-beads) were measured to assess the stability of the PPase-bead complexes.

100 μL of PPase-beads (in storage buffer) or respective supernatants (storage buffer alone) were added to 50 μL 500 mM Tris-HCl pH 9.0, 1 μL 1 M MgCl₂, and 344 μL water for injection. After adding 5 μL of 200 mM pyrophosphate (PPi), the reactions were mixed and incubated for 10 minutes at 25° C. The reaction was stopped by adding 500 μL of 40 mM EDTA.

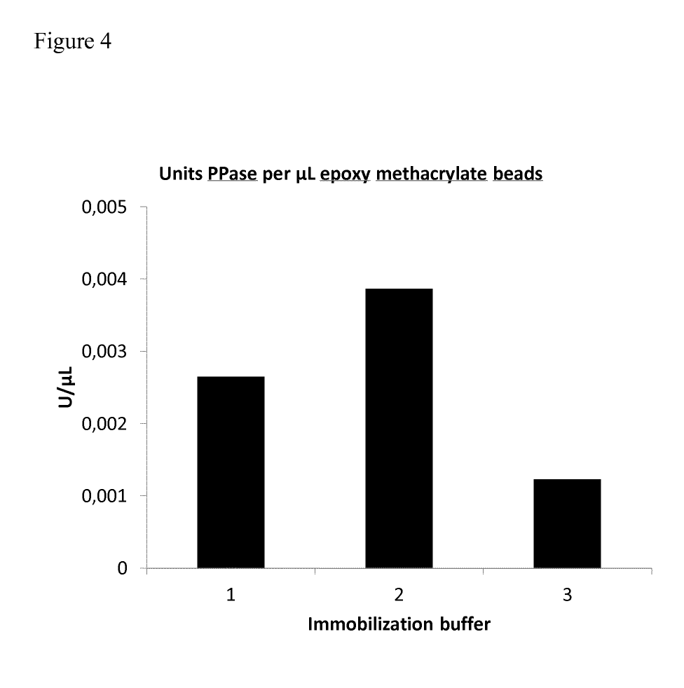

2 μL of the respective samples were used to perform a phosphate colorimetric assay (commercially available kit, according to the manufacturer's instructions). The results are shown in FIG. 4 and described below.

### 4. Results:

Using the above described experimental procedure, native E. coli PPase (SEQ ID NO: 1) was successfully immobilized on epoxy methacrylate beads. It can reasonably be expected that the same immobilization method is also applicable to PPases of other organisms, such as of Thermus thermophilus and Thermus aquaticus. As also explained above, the skilled person knows how to determine whether thiol groups, e.g., cysteine residues, are present at positions suitable for immobilization onto a solid support. Moreover, new cysteine residues can be attached to the C-terminus via a linker or directly or introduced into the amino acid sequence at a desired position as described above. Hence, the above method will even be applicable for PPases having no cysteine residue or not having a cysteine residue at a suitable position in the native amino acid sequence.

The results in FIG. 4 show that pyrophosphatase activity (expressed as units PPase per μL) was measured in PPase-bead material. This data demonstrates that the used immobilization procedure via was successful and that the enzymatic activity of the PPase was not destroyed. The reaction conditions of immobilization were chosen to obtain PPase immobilized via stable covalent thioether linkages. This type of covalent bond is particularly useful for the application of PPase-beads in RNA in vitro transcription (IVT) reaction, because thio ether linkages are insensitive to reducing agents commonly present in conventional IVT buffers such as DTT. The long term activity and long term stability of the obtained PPase-beads was analyzed in the example below (see Example 2).

### Example 2: Long-Term Activity and Stability of Immobilized PPase

The goal of this experiment was to evaluate long-term stability of the immobilized PPase obtained according to Example 1. The long-term activity of immobilized PPase was tested according to the colorimetric assay explained in Example 1. In addition to the activity of immobilized PPase, the stability of the PPase-bead complexes was evaluated.

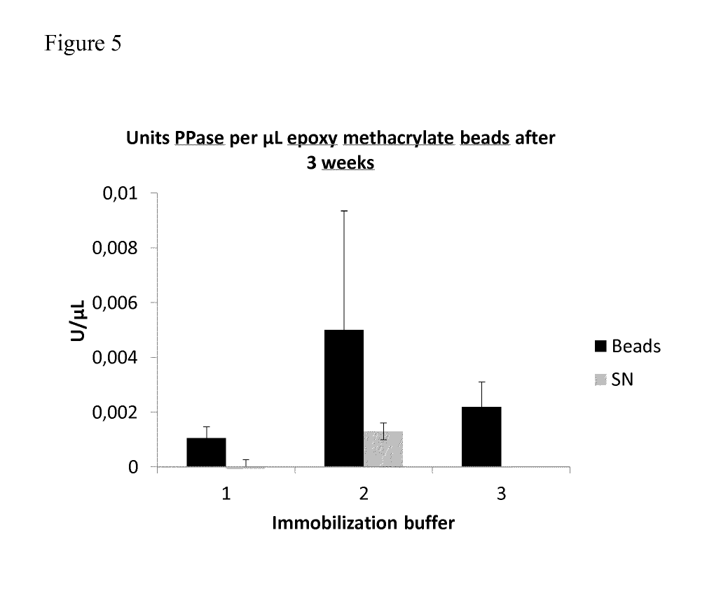

The enzymatic activity of PPase-beads and respective supernatants (storage buffer without beads) was tested at 3 weeks post immobilization. The results of the analysis are shown in FIG. 5.

### Results:

The results show that the enzymatic activity of PPase-beads did not decrease over a storage period of 3 weeks. In addition, the results show that the PPase was stably immobilized, because the measured respective supernatant showed considerably less enzymatic activity. The results highlight that the PPase-beads can be used as reusable catalysts in various enzymatic reactions, e.g. in the in vitro synthesis of nucleic acids.

### Example 3: Immobilization of Mutated E. coli and T. aquaticus PPase

In the present example, 3 mg of each purified recombinant mutated PPases derived from E. coli (SEQ ID NOs: 2 to 9), Thermus thermophilus (SEQ ID NOs: 16 to 21) and Thermus aquaticus (SEQ ID NO: 11 to 14) are immobilized via introduced cysteine residues located directly at the C-terminus or via a C-terminal glycine rich linker element (codon optimization, gene synthesis sub cloning, protein expression and protein purification performed by a commercial provider).

Respective proteins are transferred to 10 mL coupling buffer (0.1 M Tris-HCl pH 7.5, 0.5 M NaCl, 1 mM EDTA). EDTA is added to the buffer to remove trace amounts of heavy metal ions, which may catalyze oxidation of thiols. De-gassing of the buffer is performed to avoid oxidation of free thiol groups. The final concentration of all proteins in coupling buffer is about 300 μg/mL.

### Coupling of Mutant PPase Protein to a Maleimide Activated Support:

Respective recombinant mutant PPases are coupled on HiTrap columns that have been pre-packed with 5 mL bed volumes of maleimide activated agarose (PureCube, Cube Biotech). The HiTrap column is connected to an input and an output tank. The flow is adjusted to 5 cm/h using a peristaltic pump.

First the column is washed 3 times with coupling buffer, with a 10-fold excess of buffer to resin bed volume. Then, the protein solution is used for coupling. With a flow-through rate of approximately 5 cm/h, coupling is allowed to happen for 2 hours. After coupling occurred, the column is washed three times with coupling buffer at a 10-fold excess of buffer to resin bed volume. After washing, the flow through is analyzed for trace protein using a Nano Drop 2000 at an absorbance wavelength of 280 nm. If coupling efficiency is less than desired, the flow-through is recycled from the output tank into the input tank onto the column for additional rounds to achieve the desired coupling efficiency (>50%). Next, excess reactive sites are blocked by washing the resin with 50 mM cysteine (in coupling buffer) for 30 min, followed by three additional washes with 25 mL coupling buffer/Triton-X.

Next, the resin is equilibrated two times with 15 mL storage buffer (50 mM Tris-HCl, 5 mM KCl, 1 mM MgCl₂, pH 8, without DTT) for 10 minutes.

The obtained PPase-beads (E. coli PPase-beads and T. aquaticus PPase-beads) are used for RNA in vitro transcription according to Example 4.

### Example 4: RNA In Vitro Transcription Using PPase-Beads

For the present example, a DNA sequence encoding Photinus pyralis luciferase (PpLuc, SEQ ID NO: 22) was prepared by modifying the native encoding PpLuc DNA sequence by GC-optimization for stabilization. The GC-optimized PpLuc DNA sequence was introduced into a pUC19 derived vector and modified to comprise a alpha-globin-3′-UTR (muag (mutated alpha-globin-3′-UTR)), a histone-stem-loop structure, and a stretch of 70× adenosine at the 3′-terminal end (poly-A-tail). The obtained plasmid DNA is used for RNA in vitro transcription experiments.

### 1. RNA In Vitro Transcription Reaction:

The RNA in vitro transcription reaction is performed using a linear DNA template (linearized using the restriction endonuclease EcoRI according to established protocols). The reaction mixture also contains 80 mM HEPES, 2 mM spermidine, 40 mM DTT, 24 mM MgCl₂, 13.45 mM NTP mixture, 16.1 mM cap analog (e.g. m7G(5′)ppp(5′)G (m7G)) and 2500 units/mL T7 RNA polymerase. Moreover, 5 units/mL PPase-beads are added (obtained according to Example 3). Samples of the respective reactions are taken at 0 minutes, 30 minutes, 60 minutes and 90 minutes to monitor the formation of precipitations (caused by pyrophosphate; measured spectrophotometrically) and to monitor the efficiency of the RNA transcription reaction. The reactions lead to the expected positive results.

1. A composition comprising a microbial inorganic pyrophosphatase (PPase) immobilized onto a solid support via at least one thiol group of said microbial PPase.

2. The composition of claim 1, wherein the microbial PPase is a bacterial PPase, an archaeal PPase or a yeast PPase.

3. The composition of claim 2, wherein the bacterial PPase is derived from a bacterium selected from the group consisting of Escherichia coli, Thermus aquaticus, and Thermus thermophilus.

4. The composition of claim 1, wherein the microbial PPase is thermostable.

5. The composition of claim 1, wherein the microbial PPase is immobilized onto the solid support via a covalent bond.

6. The composition of claim 1, wherein the solid support comprises a reactive group selected from the group consisting of thiol, haloacetyl, pyridyl disulfide, epoxy, maleimide and mixtures thereof.

7. The composition of claim 1, wherein the solid support comprises a member selected from the group consisting of sepharose, agarose, sephadex, silica, metal and magnetic beads, methacrylate beads, glass beads, silicon, polydimethylsiloxane (PDMS), plastic materials, porous membranes, papers, alkoxysilane-based sol gels, polymethylacrylate, polyacrylamide, cellulose, monolithic supports, expanded-bed adsorbents, nanoparticles and combinations thereof.

8. The composition of claim 1, wherein the solid support is selected from the group consisting of thiol sepharose, thiopropyl sepharose, thiol-activated sephadex, thiol-activated agarose, silica-based thiol-activated matrix, silica-based thiol-activated magnetic beads, pyridyl disulfide-functionalized nanoparticles, maleimide-activated agarose, epoxy methacrylate beads and mixtures thereof.

9. The composition of claim 1, wherein the at least one thiol group of said microbial PPase is the thiol group of at least one cysteine residue of said microbial PPase.

10. The composition of claim 1, wherein the microbial PPase is immobilized onto the solid support via a bond selected from the group consisting of a disulfide bond, a thioester bond, a thioether bond and combinations thereof.

11. The composition of claim 1, wherein the microbial PPase comprises an amino acid sequence at least 80% identical to an amino acid sequence as depicted in any one of SEQ ID NOs: 1 to 21.

12. The composition of claim 1, wherein the microbial PPase is mutated.

13. The composition of claim 1, wherein the microbial PPase comprises only one cysteine residue or is mutated to comprise only one cysteine residue.

14. A method for producing a microbial PPase immobilized onto a solid support via at least one thiol group of said microbial PPase, the method comprising a step of a) contacting the microbial PPase in a reaction buffer with a solid support under conditions suitable for immobilizing the microbial PPase onto the solid support via at least one thiol group of the microbial PPase.

15-24. (canceled)

25. An immobilized microbial PPase obtained by the method of claim 14.

26. A method for enhancing a nucleic acid synthesis reaction, the method comprising performing the nucleic acid synthesis reaction in the presence of a composition of claim 1.

27-43. (canceled)

44. An enzyme reactor (1) comprising a composition of claim 1.

45-62. (canceled)

63. A kit comprising
a composition of claim 1,
a DNA or RNA polymerase, and
at least one buffer selected from the group consisting of a PPase reaction buffer, a DNA polymerase reaction buffer, a RNA polymerase reaction buffer and combinations thereof.
